(19) United States
(12) Patent Application Publication
(10) Pub. No.: US 2003/0157535 A1
(43) Pub. Date: Aug. 21, 2003

(54) Identification of two principal mutations in ion channels associated with idiopathic generalised epilepsies

(76) Inventor: Samuel Frank Berkovic, Caulfield North (AU)

Correspondence Address: JENKINS & WILSON, PA, 3100 TOWER BLVD, SUITE 1400, DURHAM, NC, 27707, US

(21) Appl. No.: 10/332,598

(22) PCT Filed: Jul. 18, 2001
(86) PCT No.: PCT/AU01/00872

(30) Foreign Application Priority Data
Jul. 18, 2000 (AU) PA 8826

(51) Int. Cl. C12Q 1/68

(52) U.S. Cl. 435/6

(57) ABSTRACT

A method for identifying the molecular defects responsible for the idiopathic generalised epilepsies (IGE), comprising the steps of: 1) providing sequence information for ion channel subunits; 2) screening a nucleic acid or peptide isolated from a patient affected by an IGE for molecular defects in the ion channel subunits in order to identify two principal defects associated with the IGE; and 3) correlating the two principal molecular defects identified with clinical observations in order to establish the combination of mutant subunits involved in the IGE.

### TECHNICAL FIELD

The present invention is concerned with mutations in proteins having biological functions as ion channels and, more particularly, with such mutations where they are associated with idiopathic generalized epilepsies (IGE).

### BACKGROUND ART

The molecular genetic era has resulted in spectacular advances in classification, diagnosis and biological understanding of numerous inherited neurological disorders including muscular dystrophies, familial neuropathies and spinocerebellar degenerations. These disorders are all uncommon or rare and have simple Mendelian inheritance. In contrast, common neurological diseases like epilepsy, migraine, and multiple sclerosis have complex inheritance where they are determined by multiple genes sometimes interacting with environmental influences. Molecular genetic advances in disorders with complex inheritance have been far more modest to date (Todd, 1999).

Most of the molecular genetic advances have occurred by a sequential three stage process. First a clinically homogeneous disorder is identified and its mode of inheritance determined. Second, linkage analysis is performed on carefully characterized clinical populations with the disorder. Linkage analysis is a process where the chromosomal localization of a particular disorder is narrowed down to approximately 0.5% of the total genome. Knowledge of linkage imparts no intrinsic biological insights other than the important clue as to where to look in the genome for the abnormal gene. Third, strategies such as positional cloning or the positional candidate approach are used to identify the aberrant gene and its specific mutations within the linked region (Collins, 1995).

Linkage studies in disorders with complex inheritance have been bedevilled by negative results and by failure to replicate positive findings. A sense of frustration permeates current literature in the genetics of complex disorders. Carefully performed, large scale studies involving hundreds of sibpairs in disorders including multiple sclerosis and diabetes have been essentially negative (Bell and Lathrop, 1996; Lernmark and Ott, 1998). An emerging view is that such disorders are due to the summation of many genes of small effect and that identification of these genes may only be possible with very large-scale association studies. Such studies on a genome-wide basis are currently impossible due to incomplete marker sets and computational limitations.

The idiopathic generalized epilepsies (IGE) are the most common group of inherited human epilepsy and do not have simple inheritance. Like other complex disorders, linkage studies in IGE have generated controversial and conflicting claims. Previous authors have suggested the possibility of multifactorial, polygenic, oligogenic or two locus models for the disease (Andermann, 1982; Doose and Baier, 1989; Greenberg et al., 1988; 1992; Janz et al., 1992).

Two broad groups of IGE are now known—the classical idiopathic generalized epilepsies (Commission on Classification and Terminology of the International League Against Epilepsy, 1989) and the newly recognized genetic syndrome of generalized epilepsy with febrile seizures plus (GEFS+) (Scheffer and Berkovic, 1997; Singh et al., 1999).

The classical IGEs are divided into a number of clinically recognizable but overlapping sub-syndromes including childhood absence epilepsy, juvenile absence epilepsy, juvenile myoclonic epilepsy etc (Commission on Classification and Terminology of the International League Against Epilepsy, 1989; Roger et al., 1992). The sub-syndromes are identified by age of onset and the pattern of seizure types (absence, myoclonus and tonic-clonic). Some patients, particularly those with tonic-clonic seizures alone do not fit a specifically recognized sub-syndrome. Arguments for regarding these as separate syndromes, yet recognizing that they are part of a neurobiological continuum, have been presented previously (Berkovic et al. 1987; 1994; Reutens and Berkovic, 1995).

GEFS⁺ was originally recognized through large multi-generation families and comprises a variety of sub-syndromes. Febrile seizures plus (FS⁺) is a sub-syndrome where children have febrile seizures occurring outside the age range of 3 months to 6 years, or have associated febrile tonic-clonic seizures. Many family members have a phenotype indistinguishable from the classical febrile convulsion syndrome and some have FS⁺ with additional absence, myoclonic, atonic, or complex partial seizures. The severe end of the GEFS⁺ spectrum includes myoclonic-astatic epilepsy.

The cumulative incidence for epilepsy by age 30 years (proportion suffering from epilepsy at some time) is 1.5% (Hauser et al., 1993). Accurate estimates for the cumulative incidence of the IGEs are unavailable. In epidemiological studies where attempts are made to subclassify epilepsies, rather few cases of IGE are diagnosed, and many cases are unclassified. This is probably because cases are rarely directly examined by epileptologists. In clinic- or office-based series seen by experts, most cases are classifiable and IGEs account for about 25% of cases. This suggests that about 0.3% of the population suffer from IGE at some time.

In outbred populations many patients with classical IGE appear to be sporadic as siblings and parents are usually unaffected. Systematic EEG studies on clinically unaffected family members show an increase in age-dependent occurrence of generalized epileptiform discharges compared to controls. In addition, to the approximate 0.3% of the population with clinical IGE, systematic EEG studies suggest that about 1% of healthy children have generalized epileptiform discharges while awake (Cavazutti et al., 1980; Okubo et al., 1994).

Approximately 5-10% of first degree relatives of classical IGE probands have seizures with affected relatives usually having IGE phenotypes or febrile seizures. While nuclear families with 2-4 affected individuals are well recognized and 3 generation families or grandparent-grandchild pairs are occasionally observed (Italian League Against Epilepsy Genetic Collaborative Group, 1993), families with multiple affected individuals extending over 4 or more generations are exceptionally rare.

For GEFS⁺, however, a number of large multi-generation families showing autosomal dominant inheritance with incomplete penetrance are known. Similar to classical IGE, analysis of sporadic cases and small families with GEFS⁺ phenotypes does not suggest simple Mendelian inheritance. Indeed, bilineal inheritance, where there is a history of epilepsy on maternal and paternal sides, is observed in both GEFS⁺ and classical IGE families (Singh et al., 1999; Italian League Against Epilepsy Genetic Collaborative Group, 1993).

Within single families with classical IGE or GEFS⁺, affected individuals often have different sub-syndromes. The closer an affected relative is to the proband, the more similar are their sub-syndromes, and siblings often have similar sub-syndromes (Italian League Against Epilepsy Genetic Collaborative Group, 1993). Less commonly, families are observed where most, or all, known affected individuals have one classical IGE sub-syndrome such as childhood absence epilepsy or juvenile myoclonic epilepsy (Italian League Against Epilepsy Genetic Collaborative Group, 1993).

Importantly, sub-syndromes are identical in affected monozygous twins with IGE. In contrast, affected dizygous twins, may have the same or different sub-syndromes. Classical IGE and GEFS⁺ sub-syndromes tend to segregate separately (Singh et al., 1999).

In some inbred communities, pedigree analysis strongly suggests recessive inheritance for juvenile myoclonic epilepsy and other forms of IGE (Panayiotopoulos and Obeid, 1989; Berkovic et al., 2000). In such families, sub-syndromes are much more similar in affected siblings than in affected sib-pairs from outbred families. Recently, a family with an infantile form of IGE with autosomal recessive inheritance, confirmed by linkage analysis, was described in Italy (Zara et al., 2000).

Most work on the molecular genetics of classical IGEs has been done on the sub-syndrome of juvenile myoclonic epilepsy where a locus in proximity or within the HLA region on chromosome 6p was first reported in 1988 (Greenberg et al., 1988). This finding was supported by two collaborating laboratories, in separate patient samples, and subsequently three groups provided further evidence for a 6p locus for juvenile myoclonic epilepsy in some, but not all, of their families. However, genetic defects have not been found and the exact locus of the gene or genes, in relationship to the HLA region, remains controversial. Strong evidence for linkage to chromosome 6 also comes from a study of a single large family with juvenile myoclonic epilepsy, but in this pedigree the locus is well outside the HLA region. A locus on chromosome 15q has also been suggested for juvenile myoclonic epilepsy, but was not confirmed by two other studies.

In general, the results of studies of the putative chromosomal 6p locus in the HLA region in patients with absence epilepsies or other forms of idiopathic generalized epilepsies have been negative. The major exception is that study of probands with tonic-clonic seizures on awakening, a sub-syndrome closely related to juvenile myoclonic epilepsy, suggests linkage to 6p.

Linkage for classical remitting childhood absence epilepsy remains elusive, but in a family with persisting absence evolving into a juvenile myoclonic epilepsy phenotype, linkage to chromosome 1p has been claimed. An Indian pedigree with persisting absence and tonic-clonic seizures may link to 8q24. Linkage to this region was also suggested by a non-parametric analysis in IGE, irrespective of subsyndrome, but was not confirmed in another study. Other loci for IGEs that have been reported in single studies include 3p14, 8p, 18 and possibly 5p. The unusual example of recessively inherited infantile onset IGE described in Italy maps to 16p in a single family.

Thus, like most disorders with complex inheritance, the literature on genetics of classical IGEs is confusing and contradictory. Some, and perhaps much, of this confusion is due to heterogeneity, with the likelihood of a number of loci for IGEs. The studies reviewed above were principally performed on multiple small families, so heterogeneity within and between samples is probable. Whether all, some, or none of the linkages reported above will be found to harbour relevant genes for IGE remains to be determined. Most of the studies reviewed above used analysis methods assuming Mendelian inheritance, an assumption that is not correct for outbred communities. Some studies used multiple models (autosomal recessive, autosomal dominant). Although parametric linkage analysis may be reliable in some circumstance of analyzing complex disease, it can lead to spurious findings as highlighted by the literature on linkage in major psychoses (Risch and Botstein, 1996).

In so far as GEFS⁺ is concerned, linkage analysis on rare multi-generation large families with clinical evidence of a major autosomal dominant gene have demonstrated loci on chromosomes 19q and 2q. Both the 19q and 2q GEFS⁺ loci have been confirmed in independently ascertained large families, and genetic defects have been identified. Families linked to 19q are known and a mutation in the gene for the β1 subunit of the neuronal sodium channel (SCN1B) has been identified (Wallace et al., 1998). This mutation results in the loss of a critical disulphide bridge of this regulatory subunit and causes a loss of function in vitro. Families linked to 2q are also known and mutations in the pore-forming ax subunit of the neuronal sodium channel (SCN1A) have been identified (Australian provisional patent PR2203; Wallace et al., 2001b; Escayg et al., 2000). Studies on the more common small families with GEFS⁺ have not revealed these or other mutations to date.

In addition to the SCN1B and SCN1A mutations in GEFS⁺, four other gene defects have been discovered for human idiopathic epilepsies through the study of large families. Mutations in the alpha-4 subunit of the neuronal nicotinic acetylcholine receptor (CHRNA4) occur in the focal epilepsy syndrome of autosomal dominant nocturnal frontal lobe epilepsy (Australian patent AU-B-56247/96; Steinlein et al., 1995). Mutations in the gamma-2 subunit of the GABA_{A} receptor (GABRG2) have been identified in childhood absence epilepsy, febrile seizures (including febrile seizures plus) and myoclonic epilepsy (PCT/AU01/00729; Wallace et al., 2001a). Finally, mutations in two potassium channel genes (KCNQ2 and KCNQ3) were identified in benign familial neonatal convulsions (Singh et al., 1998; Biervert et al., 1998; Charlier et al., 1998). Although initially regarded as a special form of IGE, this unusual syndrome is probably a form of inherited focal epilepsy.

Further to these studies, mutations in other genes have been identified to be causative of epilepsy. These include mutations in the beta-2 subunit (CHRNB2) of the neuronal nicotinic acetylcholine receptor (PCT/AU01/00541; Phillips et al., 2001) and the delta subunit (GABRD) of the GABA_{A} receptor (PCT/AU01/00729).

A number of mouse models approximating human IGE are known. These mice mutants have ataxia in addition to generalized spike-and-wave discharges with absences or tonic-clonic seizures. Recessive mutations in calcium channel subunit genes have been found in lethargic (CACNB4), tottering/leaner (CACNA1A), and stargazer (CACNG2) mutants. The slow-wave epilepsy mouse mutant has a mutation in the sodium/hydrogen exchanger gene, which may have important downstream effects on pH-sensitive ion channels.

The human and mouse literature is now suggesting that the idiopathic epilepsies comprise a family of channelopathies with mutations in ion channel subunits of voltage-gated (eg SCN1A, SCN1B, KCNQ2, KCNQ3) or ligand-gated (eg CHRNA4, CHRNB2, GABRG2, GABRD) types. These channels are typically comprised of a number of subunits, specified by genes on different chromosomes. The stoichiometry and conformation of ion channel subunits are not yet well understood, but many have multiple subunits in a variety of combinations.

### DISCLOSURE OF THE INVENTION

The present invention arises from a new genetic model for the idiopathic generalised epilepsies (IGEs) in which it is postulated that IGEs are due to the combination of two mutations in ion channels, in particular, due to separate mutations in each of two subunits of the multiple subunits of ion channels.

While not wishing to be bound by theory it is believed that:

1. Most classical IGE and GEFS⁺ cases are due to the combination of two mutations in multi-subunit ion channels. These are typically point mutations resulting in subtle change of function. The critical postulate is that two mutations, usually, but not exclusively, in different subunit alleles (“digenic model”), are required for clinical expression of IGE. Further, we posit that

a) A number of different mutated subunit pairs can be responsible for IGE. Combinations of two mutated subunits lead to an IGE genotype with 30% penetrance.

b) The total allele frequency of mutated subunits is −8%. It has been calculated below that approximately 15% of the population has one or more mutated subunit genes and 1% have two or more mutated subunits.

c) Sub-syndromes are principally determined by the specific combination of mutated subunit pairs, although one or more other genes of smaller effect may modify the phenotype.

d) Mutated subunit combinations that cause classical IGEs are largely separate from those that cause GEFS⁺, although some subunits may be involved in both syndromes.

e) Individuals with single ‘change of function’ mutations would not have IGE, but such mutations may contribute to simple febrile seizures, which are observed with increased frequency in relatives of IGE probands.

2. Subunit mutations with more severe functional consequences (eg breaking a disulphide bridge in SCN1B or amino acid substitution in the pore forming regions of SCN1A for GEFS⁺) cause autosomal dominant generalized epilepsies with a penetrance of 60-90%. The precise sub-syndromes in GEFS⁺ are determined by minor allelic variation or mutations in other ion channel subunits. Such “severe” mutations are rare (allele frequency <0.01%) and are infrequent causes of GEFS⁺. They very rarely, or perhaps never, cause classical IGE.

According to one aspect of the present invention there is provided a method for identifying the molecular defects responsible for the idiopathic generalised epilepsies (IGE), comprising the steps of:

(1) providing sequence information for ion channel subunits;

(2) screening a nucleic acid or peptide isolated from a patient affected by an IGE for molecular defects in the ion channel subunits in order to identify two principal defects associated with the IGE; and

(3) correlating the two principal molecular defects identified with clinical observations in order to establish the combination of mutant subunits involved in the IGE.

In order to characterise the idiopathic generalised epilepsies a plurality of patients with different forms of IGE may be screened and the two principal molecular defects identified in each patient or patients with a particular IGE correlated with clinical observations. In this way the combination of the two principal molecular defects involved in each IGE may be established.

It will be appreciated, however, that single, severe mutations are infrequent causes of GEFS⁺, and on these occasions it will not prove possible to identify two specific mutations associated with the sub-syndrome.

Accordingly, in a preferred form of the invention the process comprises the further step of determining whether two principal defects may be established to be associated with a sub-syndrome, particularly of GEFS⁺, but also of classical IGE in the event that any such sub-syndrome is caused by a single mutation.

It will also be appreciated that further molecular defects which modify the phenotype may be identified.

Accordingly, in a preferred form of the invention there is provided the further step of identifying additional molecular defects in genes of smaller effect which may modify the IGE phenotype.

In a further aspect of the present invention there is provided a method for establishing the loci of molecular defects of ion channel subunit genes responsible for the idiopathic generalised epilepsies (IGE), comprising the steps of:

1) obtaining a DNA sample from a patient affected with epilepsy;

2) comparing the DNA, including that of the ion channel subunit genes from said DNA sample with that from a corresponding wild type DNA sample;

3) identifying those patient DNA samples in which two loci segregate with the disease; and

4) determining the location of the two abnormal loci

Typically large families would be identified in which two abnormal alleles co-segregate by chance. Careful clinical phenotyping in these large families, together with a linkage analysis, allows identification of the abnormal segregating loci. These families are investigated further to identify the relevant mutations present in the ion channel subunit genes present at these loci which give rise to a particular form of IGE.

It will be appreciated that for each molecular defect one can provide an isolated mammalian DNA coding for a protein having a biological function as part of an ion channel in a mammal, wherein a mutation event selected from the group consisting of point mutations, deletions, insertions and rearrangements has occurred so as to affect the functioning of the ion channel. In the model, there is provided a second isolated mammalian DNA coding for a protein having a biological function as part of an ion channel in a mammal, wherein a mutation event selected from the group consisting of point mutations, deletions, insertions and rearrangements has occurred so as to affect the functioning of the ion channel. The cumulative effect of the mutations in each isolated mammalian DNA molecule in vivo is to produce an idiopathic generalized epilepsy in said mammal. The mutations may be in mammalian DNA coding for protein subunits belonging to the same ion channel or may be in mammalian DNA coding for protein subunits that belong to a different ion channel.

Typically the mutation is a point mutation and the ion channels are voltage-gated channels such as a sodium, potassium, calcium or chloride channels or are ligand-gated channels such as members of the nAChR/GABA super family of receptors, or a functional fragment or homologue thereof.

Mutations may include those in non-coding regions of the ion channel subunits (eg mutations in the promoter region which affect the level of expression of the subunit gene or mutations in intronic sequences which affect the correct splicing of the subunit during mRNA processing). Mutations may also, and more preferably, will be in coding regions of the ion channel subunits (eg nucleotide mutations may give rise to an amino acid change in the encoded protein or nucleotide mutations that do not give rise to an amino acid change but may affect the stability of the mRwk).

Mutation combinations may be selected from, but are not restricted to, those identified in Tables 1A and 1B, those disclosed in our previous patent applications (eg AU-B-56247/96; PCT/AU01/00541; PCT/AU01/00729; Australian provisional patents PR2203 and PR4922), the contents of which are incorporated herein by reference, or those represented in the literature and those yet to be identified. Each of these mutations can be used to confirm the digenic hypothesis of this invention. The novel mutations identified in Tables 1A and 1B are described in our Australian provisional patent application entitled “Mutations in Ion Channels” filed concurrently herewith, the contents of which are incorporated herein by reference.

Nucleotide sequences containing the molecular defects described above can be engineered using methods accepted in the art for a variety of purposes. These include, but are not limited to, modification of the cloning, processing, and/or expression of the gene product. PCR reassembly of gene fragments and the use of synthetic oligonucleotides allow the engineering of the nucleotide sequences of the present invention. For example, oligonucleotide-mediated site-directed mutagenesis can introduce further mutations that create new restriction sites, alter expression patterns and produce splice variants etc.

As a result of the degeneracy of the genetic code, a number of polynucleotide sequences, some that may have minimal similarity to the polynucleotide sequences of any known and naturally occurring gene, may be produced. Thus, the invention includes each and every possible variation of a polynucleotide sequence that could be made by selecting combinations based on possible codon choices. These combinations are made in accordance with the standard triplet genetic code as applied to the polynucleotide sequences of the present invention, and all such variations are to be considered as being specifically disclosed.

The DNA molecules may be cDNA, genomic DNA, synthetic forms, and mixed polymers, both sense and antisense strands, and may be chemically or biochemically modified, or may contain non-natural or derivatised nucleotide bases as will be appreciated by those skilled in the art. Such modifications include labels, methylation, intercalators, alkylators and modified linkages. In some instances it may be advantageous to produce nucleotide sequences possessing a substantially different codon usage than that of the polynucleotide sequences of the present invention. For example, codons may be selected to increase the rate of expression of the peptide in a particular prokaryotic or eukaryotic host corresponding with the frequency that particular codons are utilized by the host. Other reasons to alter the nucleotide sequence without altering the encoded amino acid sequences include the production of RNA transcripts having more desirable properties, such as a greater half-life, than transcripts produced from the naturally occurring mutated sequence.

The invention also encompasses production of DNA sequences embodying the molecular defects described above entirely by synthetic chemistry. Synthetic sequences may be inserted into expression vectors and cell systems that contain the necessary elements for transcriptional and translational control of the inserted coding sequence in a suitable host. These elements may include regulatory sequences, promoters, 5′ and 3′ untranslated regions and specific initiation signals (such as an ATG initiation codon and Kozak consensus sequence) which allow more efficient translation of sequences encoding the polypeptides of the present invention. In cases where the complete coding sequence, including the initiation codon and upstream regulatory sequences, are inserted into the appropriate expression vector, additional control signals may not be needed. However, in cases where only coding sequence, or a fragment thereof, is inserted, exogenous translational control signals as described above should be provided by the vector. Such signals may be of various origins, both natural and synthetic. The efficiency of expression may be enhanced by the inclusion of enhancers appropriate for the particular host cell system used (Scharf et al., 1994).

Nucleic acid molecules that are the complements of the sequences described herein may also be prepared.

The present invention allows for the preparation of purified polypeptide or protein from the polynucleotides of the present invention, or variants thereof. In order to do this, host cells may be transformed with DNA molecules encoding two mutant ion channel subunits, whether novel or not. If the two mutant subunits form a part of the same ion channel a receptor protein containing two mutant subunits may be isolated. If the mutant subunits are subunits of different ion channels the host cells will express two mutant receptor proteins. Typically said host cells are transfected with an expression vector comprising a DNA molecule or molecules encoding two mutant ion channel subunits. A variety of expression vector/host systems may be utilized to contain and express sequences encoding polypeptides of the invention. These include, but are not limited to, microorganisms such as bacteria transformed with plasmid or cosmid DNA expression vectors; yeast transformed with yeast expression vectors; insect cell systems infected with viral expression vectors (e.g., baculovirus); or mouse or other animal or human tissue cell systems. Mammalian cells can also be used to express a protein using a vaccinia virus expression system. The invention is not limited by the host cell or vector employed.

The polynucleotide sequences, or variants thereof, of the present invention can be stably expressed in cell lines to allow long term production of recombinant proteins in mammalian systems. Sequences encoding the polypeptides of the present invention can be transformed into cell lines using expression vectors which may contain viral origins of replication and/or endogenous expression elements and a selectable marker: gene on the same or on a separate vector. The selectable marker confers resistance to a selective agent, and its presence allows growth and recovery of cells which successfully express the introduced sequences. Resistant clones of stably transformed cells may be propagated using tissue culture techniques appropriate to the cell type.

In addition, a host cell strain may be chosen for its ability to modulate expression of the inserted sequences or to process the expressed protein in the desired fashion. Such modifications of the polypeptide include, but are not limited to, acetylation, glycosylation, phosphorylation, and acylation. Post-translational cleavage of a “prepro” form of the protein may also be used to specify protein targeting, folding, and/or activity. Different host cells having specific cellular machinery and characteristic mechanisms for post-translational activities (e.g., CHO or HeLa cells), are available from the American Type Culture Collection (ATCC) and may be chosen to ensure the correct modification and processing of the foreign protein.

When large quantities of the protein product of the gene are needed, such as for antibody production, vectors which direct high levels of expression of this protein may be used, such as those containing the T5 or T7 inducible bacteriophage promoter. The present invention also includes the use of the expression systems described above in generating and isolating fusion proteins which contain important functional domains of the protein. These fusion proteins are used for binding, structural and functional studies as well as for the generation of appropriate antibodies.

In order to express and purify the protein as a fusion protein, the appropriate cDNA sequence is inserted into a vector which contains a nucleotide sequence encoding another peptide (for example, glutathionine succinyl transferase). The fusion protein is expressed and recovered from prokaryotic or eukaryotic cells. The fusion protein can then be purified by affinity chromatography based upon the fusion vector sequence. The desired protein is then obtained by enzymatic cleavage of the fusion protein.

Fragments of the polypeptides of the present invention may also be produced by direct peptide synthesis using solid-phase techniques. Automated synthesis may be achieved by using the ABI 431A Peptide Synthesizer (Perkin-Elmer). Various fragments of this protein may be synthesized separately and then combined to produce the full length molecule.

According to another aspect of the present invention there is provided an isolated mammalian polypeptide having a biological function as an ion channel in a mammal, wherein a mutation event selected from the group consisting of substitutions, deletions, truncations, insertions and rearrangements has occurred in each of two subunits so as to affect the functioning of the ion channel, the cumulative effect of the mutations in each isolated mammalian polypeptide in vivo being to produce an idiopathic generalized epilepsy in said mammal. In the digenic model it will be appreciated that the mutations may be in polypeptide subunits belonging to the same ion channel as described above, but may also be in polypeptide subunits that belong to different ion channels.

Typically the mutation is an amino acid substitution and the ion channel is a voltage-gated channel such as a sodium, potassium, calcium or chloride channel or a ligand-gated channel such as a member of the nAChR/GABA super family of receptors, or a functional fragment or homologue thereof.

Mutation combinations may be selected from, but are not restricted to, those represented in Tables 1A and 1B, those disclosed in our previous patent applications (eg AU-B-56247/96; PCT/AU01/00541; PCT/AU01/00729; Australian provisional patents PR2203 and PR4922), the contents of which are incorporated herein by reference, or those represented in the literature and those yet to be identified. Each of these mutations can be used to confirm the digenic hypothesis of this invention. As previously indicated the novel mutations identified in Tables 1A and 1B are described in our Australian provisional patent application entitled “Mutations in Ion Channels” filed concurrently herewith, the contents of which are incorporated herein by reference.

According to still another aspect of the present invention there is provided a method of preparing a polypeptide, said polypeptide being a mutant ion channel, comprising the steps of:

(1) culturing host cells transfected with an expression vector comprising a DNA molecule as described above under conditions effective for polypeptide production; and

(2) harvesting the mutant ion channel.

The mutant ion channel subunit may be allowed to assemble with other subunits constituting the channel that are either wild-type or themselves mutant subunits, whereby the assembled ion channel is harvested. In the latter case, the molecule may be used to confirm the digenic model.

Substantially purified protein or fragments thereof can then be used in further biochemical analyses to establish secondary and tertiary structure for example by X-ray crystallography of crystals of the proteins or of the assembled ion channel or by nuclear magnetic resonance (NMR). Determination of structure allows for the rational design of pharmaceuticals to interact with the ion channel as a whole or through interaction with a specific subunit protein, alter the overall ion channel protein charge configuration or charge interaction with other proteins, or to alter its function in the cell.

It will be appreciated that the mutant ion channel subunits included as part of the present invention will be useful in further applications which include a variety of hybridisation and immunological assays to screen for and detect the presence of either a normal or mutated gene or gene product. The invention enables therapeutic methods for the treatment of epilepsy and also enables methods for the diagnosis of epilepsy.

Accordingly, in still another aspect of the present invention there is provided a method for the treatment of an IGE, comprising administering to a patient with two principal molecular defects in their ion channel subunits which are causative of the IGE, one or more therapeutic agents which, singly or collectively, overcome or ameliorate the effect of the two principal molecular defects.

As will be further described below, the or each therapeutic agent is selected from the group consisting of a wild-type ion channel subunit polypeptide, a nucleic acid encoding a wild-type ion channel subunit, a nucleic acid encoding the complement of an ion channel subunit gene, and an agonist, modulator or antagonist of an ion channel subunit containing one of the two principal molecular defects.

In a further aspect of the present invention there is provided the use of a wild-type ion channel subunit polypeptide, a nucleic acid encoding a wild-type ion channel subunit, a nucleic acid encoding the complement of an ion channel subunit gene, or an agonist, modulator or antagonist of an ion channel subunit containing one of two principal molecular defects causative of an IGE, in the preparation of a medicament for the treatment of the IGE.

In one form of the invention there is provided a method of treating an IGE comprising administering a wild-type ion channel polypeptide or ion channel subunit polypeptide as described above, or an agonist or modulator of the ion channel, when the channel contains a mutation in any one of the subunits comprising the channel, said mutation being causative of the IGE when expressed in combination with a second mutation in a subunit of the same or different ion channel, to a subject in need of such treatment. All therapeutic methods described below may address correcting both mutations or may be directed at correcting a single mutation.

In still another form of the invention there is provided the use of a selective agonist or modulator of an ion channel when the channel contains a mutation in any one of the subunits comprising the channel, said mutation being causative of an IGE when expressed in combination with a second mutation in a subunit of the same or different ion channel, in the manufacture of a medicament for the treatment of the disorder.

Proteins or peptides which represent the appropriate ion channel subunits can be supplied to cells which contain the corresponding mutant or missing ion channel subunits. Ion channel subunit proteins can be produced as described above and can be introduced into cells by microinjection or by use of liposomes as another example Some molecules may be taken up by cells actively or by diffusion. Supply of proteins or peptides with the appropriate ion channel subunit activity to cells deficient in such subunits should lead to partial reversal of epilepsy.

Other molecules with ion channel subunit activity (for example drugs or organic compounds) may also be used for therapy.

A pharmaceutical composition comprising one or more substantially purified ion channel subunits or other molecules as described above and a pharmaceutically acceptable carrier may be administered.

Pharmaceutical compositions in accordance with the present invention are prepared by mixing the appropriate ion channel subunits, or active fragments or variants thereof, or other molecules, having the desired degree of purity, with acceptable carriers, excipients, or stabilizers which are well known. Acceptable carriers, excipients or stabilizers are nontoxic at the dosages and concentrations employed, and include buffers such as phosphate, citrate, and other organic acids; antioxidants including absorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitrol or sorbitol; salt-forming counterions such as sodium; and/or nonionic surfactants such as Tween, Pluronics or polyethylene glycol (PEG).

The appropriate ion channel subunit genes or fragments thereof may be employed in gene therapy methods in order to increase the amount of the expression products of the genes in mutated cells.

Alternatively, the appropriate ion channel subunit genes, or fragments thereof, may be delivered to affected cells in a form that can be taken up and can code for sufficient protein to provide effective function.

The method of treating an IGE may comprise administering an agonist or modulator of a mutated ion channel and/or an isolated DNA molecule as described above or a wild-type ion channel or ion channel subunit, to a subject in need of such treatment.

There is also provided the use of an agonist or modulator of a mutated ion channel and/or isolated DNA molecule as described above or a wild-type ion channel or ion channel subunit in the manufacture of a medicament for the treatment of an IGE.

Typically, vectors capable of expressing the appropriate ion channel subunits or fragments or derivatives thereof may be administered to a subject such that the genes will be expressed by the cell and remain extrachromosomal or ideally will be introduced into the cell such that it recombines with the endogenous mutant gene present in the cell. This requires a double recombination event for the correction of the gene mutation. Vectors for the introduction of genes in these ways are known in the art, and any suitable vector may be used.

Appropriate vectors for gene therapy include plasmid vectors and virus vectors including transducing retroviral vectors, adenoviruses, adeno-associated virus, vaccinia virus, papovaviruses, lentiviruses and retroviruses of avian, murine and human origin.

Gene therapy would be carried out according to accepted methods (Friedman, 1991; Culver, 1996). A vector containing a copy of the appropriate ion channel subunit gene linked to expression control elements and capable of replicating inside the cells is prepared. Alternatively the vector may be replication deficient and may require helper cells for replication and use in gene therapy. The vector is then injected into the patient and if the gene is not permanently incorporated into the genome of the target cells, the treatment may have to be repeated.

Gene transfer using non-viral methods of infection in vitro can also be used. These methods include direct injection of DNA, uptake of naked DNA in the presence of calcium phosphate, electroporation, protoplast fusion or liposome delivery. Gene transfer can also be achieved by delivery as a part of a human artificial chromosome or receptor-mediated gene transfer. This involves linking the DNA to a targeting molecule that will bind to specific cell-surface receptors to induce endocytosis and transfer of the DNA into mammalian cells. One such technique uses poly-L-lysine to link asialoglycoprotein to DNA. An adenovirus is also added to the complex to disrupt the lysosomes and thus allow the DNA to avoid degradation and move to the nucleus. Infusion of these particles intravenously has resulted in gene transfer into hepatocytes.

Gene transfer techniques which target DNA directly to the brain are preferred. For example, patients carrying ion channel subunit susceptible alleles are treated with a gene-delivery method such that some or all of their brain precursor cells receive at least one additional functional normal copy of the appropriate ion channel subunit or subunits needed. The treated individuals should have a reduced risk of disease due to the fact that the susceptible allele or alleles have been countered by the presence of the normal allele or alleles.

The method of treating an IGE may also comprise administering a selective antagonist or modulator of an ion channel or ion channel subunit, when the channel contains a mutation in a subunit comprising the channel, said mutation being causative of the IGE when expressed in combination with a second mutation in a subunit of the same or different ion channel, to a subject in need of such treatment.

There is also provided the use of a selective-antagonist or modulator of an ion channel or ion channel subunit when the ion channel contains a mutation in a subunit comprising the channel, said mutation being causative of an IGE when expressed in combination with a second mutation in a subunit of the same or different ion channel, in the manufacture of a medicament for the treatment of the IGE.

Using methods well known in the art, a mutant ion channel may be used to produce antibodies specific for the mutant channel or to screen libraries of pharmaceutical agents to identify those that specifically bind the mutant ion channel.

In one aspect, an antibody, which specifically binds to a mutant ion channel, may be used directly as an antagonist or modulator, or indirectly as a targeting or delivery mechanism for bringing a pharmaceutical agent to cells or tissues that express the mutant ion channel.

Preferably the antibody is immunologically reactive with a polypeptide as described above, but not with a wild-type ion channel or subunit thereof.

In particular, there is provided an antibody to an assembled ion channel containing a mutation in a subunit comprising the receptor, which is causative of an IGE when expressed in combination with a second mutation in a subunit of the same or different ion channel. Such antibodies may include, but are not limited to, polyclonal, monoclonal, chimeric, and single chain antibodies as would be understood by the person skilled in the art.

For the production of antibodies, various hosts including rabbits, rats, goats, mice, humans, and others may be immunized by injection with a polypeptide as described or with any fragment or oligopeptide thereof which has immunogenic properties. Various adjuvants may be used to increase immunological response and include, but are not limited to, Preund's, mineral gels such as aluminum hydroxide, and surface-active substances such as lysolecithin. Adjuvants used in humans include BCG (Bacilli Calmette-Guerin) and Corynebacterium parvum.

It is preferred that the oligopeptides, peptides, or fragments used to induce antibodies to the mutant ion channel have an amino acid sequence consisting of at least amino acids, and, more preferably, of at least 10 amino acids. It is also preferable that these oligopeptides, peptides, or fragments are identical to a portion of the amino acid sequence of the natural protein and contain the entire amino acid sequence of a small, naturally occurring molecule. Short stretches of ion channel amino acids may be fused with those of another protein, such as KLH, and antibodies to the chimeric molecule may be produced.

Monoclonal antibodies to a mutant ion channel may be prepared using any technique which provides for the production of antibody molecules by continuous cell lines in culture. These include, but are not limited to, the hybridoma technique, the human B-cell hybridoma technique, and the EBV-hybridoma technique. (For example, see Kohler et al., 1975; Kozbor et al., 1985; Cote et al., 1983; Cole et al., 1984).

Antibodies may also be produced by inducing in vivo production in the lymphocyte population or by screening immunoglobulin libraries or panels of highly specific binding reagents as disclosed in the literature. (For example, see Orlandi et al., 1989; Winter and Milstein, 1991).

Antibody fragments which contain specific binding sites for an ion channel may also be generated. For example, such fragments include, F(ab′)2 fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity. (For example, see Huse et al., 1989).

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between an ion channel and its specific antibody. A two-site, monoclonal-based immunoassay utilizing monoclonal antibodies reactive to two non-interfering ion channel epitopes is preferred, but a competitive binding assay may also be employed.

The method of treating an IGE may also comprise administering an isolated DNA molecule which is the complement (antisense) of any one of the DNA molecules described above- and which encodes a mRNA that hybridizes with the mRNA encoding a mutant ion channel subunit, to a subject in need of such treatment.

There is also provided the use of an isolated DNA molecule which is the complement of a DNA molecule of the invention and which encodes a mRNA that hybridizes with the mRNA encoding a mutant ion channel subunit, in the manufacture of a medicament for the treatment of an IGE.

Typically, a vector expressing the complement of the polynucleotides encoding the subunits constituting the ion channel may be administered to a subject in need of such treatment. Antisense strategies may use a variety of approaches including the use of antisense oligonucleotides, injection of antisense RNA and transfection of antisense RNA expression vectors. Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art. (For example, see Goldman et al., 1997).

In further embodiments, any of the agonists, proteins, antagonists, modulators, antibodies, complementary sequences or vectors of the invention may be administered in combination with other appropriate therapeutic agents. Selection of the appropriate agents may be made by those skilled in the art, according to conventional pharmaceutical principles. The combination of therapeutic agents may act synergistically to effect the treatment or prevention of epilepsy. Using this approach, therapeutic efficacy with lower dosages of each agent may be possible, thus reducing the potential for adverse side effects.

According to still another aspect of the invention, peptides of the invention, particularly purified mutant ion channel polypeptides and cells expressing these, are useful for the screening of candidate pharmaceutical agents in a variety of techniques. It will be appreciated that therapeutic agents useful in the treatment of epilepsy are likely to show binding affinity to the polypeptides of the invention. Such techniques include, but are not limited to, utilising eukaryotic or prokaryotic host cells that are stably transformed with recombinant polypeptides expressing the polypeptide or fragment, preferably in competitive binding assays. Binding assays will measure for the formation of complexes between a specific ion channel subunit polypeptide or fragment and the agent being tested, or will measure the degree to which an agent being tested will interfere with the formation of a complex between a specific ion channel subunit polypeptide or fragment and a known ligand.

Another technique for drug identification provides high-throughput screening for compounds having suitable binding affinity to the mutant ion channel polypeptides (see PCT published application WO84/03564). In this stated technique, large numbers of small peptide test compounds can be synthesised on a solid substrate and can be assayed through ion channel polypeptide binding and washing. Bound ion channel polypeptide is then detected by methods well known in the art. In a variation of this technique, purified polypeptides of the invention can be coated directly onto plates to identify interacting test compounds. In an alternative approach, high throughput screening of low molecular weight chemical libraries can be utilised.

The invention also contemplates the use of competition drug screening assays in which neutralizing antibodies capable of specifically binding the mutant ion channel compete with a test compound for binding thereto. In this manner, the antibodies can be used to detect the presence of any peptide that shares one or more antigenic determinants of the mutant ion channel.

The invention is particularly useful for screening compounds by using the polypeptides of the invention in transformed cells, transfected or injected oocytes or transgenic animals. A particular drug is added to the cells in culture or administered to a transgenic animal containing the mutant ion channel or channels and the effect on the current of the receptor is compared to the current of a cell or animal containing the wild-type ion channels. Drug candidates that alter the current to a more normal level are useful for treating or preventing epilepsy.

The polypeptides of the present invention may also be used for screening compounds developed as a result of combinatorial library technology. This provides a way to test a large number of different substances for their ability to modulate activity of a polypeptide. The use of peptide libraries is preferred (see WO 97/02048) with such libraries and their use known in the art.

A substance identified as a modulator of polypeptide function may be peptide or non-peptide in nature. Non-peptide “small molecules” are often preferred for many in vivo pharmaceutical applications. In addition, a mimic or mimetic of the substance may be designed for pharmaceutical use. The design of mimetics based on a known pharmaceutically active compound (“lead” compound) is a common approach to the development of novel pharmaceuticals. This is often desirable where the original active compound is difficult or expensive to synthesise or where it provides an unsuitable method of administration. In the design of a mimetic, particular parts of the original active compound that are important in determining the target property are identified. These parts or residues constituting the active region of the compound are known as its pharmacophore. Once found, the pharmacophore structure is modelled according to its physical properties using data from a range of sources including x-ray diffraction data and NMR. A template molecule is then selected onto which chemical groups which mimic the pharmacophore can be added. The selection can be made such that the mimetic is easy to synthesise, is likely to be pharmacologically acceptable, does not degrade in vivo and retains the biological activity of the lead compound. Further optimisation or modification can be carried out to select one or more final mimetics useful for in vivo or clinical testing.

It is also possible to isolate a target-specific antibody and then solve its crystal structure. In principle, this approach yields a pharmacophore upon which subsequent drug design can be based as described above. It may be possible to avoid protein crystallography altogether by generating anti-idiotypic antibodies (anti-ids) to a functional, pharmacologically active antibody. As a mirror image of a mirror image, the binding site of the anti-ids would be expected to be an analogue of the original receptor. The anti-id could then be used to isolate peptides from chemically or biologically produced peptide banks.

Any of the therapeutic methods described above may be applied to any subject in need of such therapy, including, for example, mammals such as dogs, cats, cows, horses, rabbits, monkeys, and most preferably, humans.

Polynucleotide sequences encoding an ion channel subunit may be used for the diagnosis of epilepsy, and the use of the DNA molecules incorporated as part of the invention in diagnosis of epilepsy, or a predisposition to epilepsy, is therefore contemplated. The novel DNA molecules incorporating the mutation events laid out in Tables 1A and 1B may be used for this purpose, and sub-syndromes of IGE may be diagnosed through identification of the two principal molecular defects.

Accordingly, in a further aspect of the present invention there is provided a method of diagnosis of an IGE, comprising the steps of:

(1) providing a DNA sample from a patient suspected of an IGE;

(2) screening the sample for molecular defects in the genes encoding ion channel subunits;

(3) establishing the presence or absence of mutations associated with IGE and, where such mutations are present, identifying the two principal mutations associated with the IGE;

(4) comparing the identified molecular defects to pre-existing data correlating molecular defects to sub-syndromes of IGE; and

(5) identifying the IGE.

The polynucleotides that may be used for diagnostic purposes include oligonucleotide sequences, genomic DNA and complementary RNA and DNA molecules. The polynucleotides may be used to detect and quantitate gene expression in biological samples. Genomic DNA used for the diagnosis may be obtained from body cells, such as those present in the blood, tissue biopsy, surgical specimen, or autopsy material. The DNA may be isolated and used directly for detection of a specific sequence or may be amplified by the polymerase chain reaction (PCR) prior to analysis. Similarly, RNA or cDNA may also be used, with or without PCR amplification. To detect a specific nucleic acid sequence, hybridisation using specific oligonucleotides, restriction enzyme digest and mapping, PCR mapping, RNAse protection, and various other methods may be employed. For instance direct nucleotide sequencing of amplification products from an ion channel subunit can be employed. Sequence of the sample amplicon is compared to that of the wild-type amplicon to determine the presence (or absence) of nucleotide differences.

It will be appreciated that identification of specific mutations in particular subunits of the various ion channels, and combinations of same, will allow for identification of the specific form of IGE.

According to a further aspect of the invention there is provided the use of a polypeptide as described above in the diagnosis of epilepsy.

Accordingly, in a further aspect of the present invention there is provided a method of diagnosis of an IGE, comprising the steps of:

(1) providing a protein sample from a patient suspected of an IGE;

(2) screening the sample for molecular defects in ion channel subunits;

(3) establishing the presence or absence of molecular defects and, where molecular defects are present, identifying two principal molecular defects associated with the IGE;

(4) comparing the identified molecular defects with pre-existing data correlating molecular defects with sub-syndromes of IGE; and

(5) identifying the IGE.

When a diagnostic assay is to be based upon proteins constituting an ion channel, a variety of approaches are possible. For example, diagnosis can be achieved by monitoring differences in the electrophoretic mobility of normal and mutant proteins that form the ion channel. Such an approach will be particularly useful in identifying mutants in which charge substitutions are present, or in which insertions, deletions or substitutions have resulted in a significant change in the electrophoretic migration of the resultant protein. Alternatively, diagnosis may be based upon differences in the proteolytic cleavage patterns of normal and mutant proteins, differences in molar ratios of the various amino acid residues, or by functional assays demonstrating altered function of the gene products.

In another aspect, antibodies that specifically bind mutant ion channels may be used for the diagnosis of epilepsy, or in assays to monitor patients being treated with a complete ion channel or agonists, antagonists, modulators or inhibitors of an ion channel. Antibodies useful for diagnostic purposes may be prepared in the same manner as described above for therapeutics. Diagnostic assays for ion channels include methods that utilize the antibody and a label to detect a mutant ion channel in human body fluids or in extracts of cells or tissues. The antibodies may be used with or without modification, and may be labelled by covalent or non-covalent attachment of a reporter molecule.

A variety of protocols for measuring the presence of mutant ion channels, including ELISAs, RIAs, and FACS, are known in the art and provide a basis for diagnosing epilepsy. The expression of a mutant ion channel or combination of mutant ion channels is established by combining body fluids or cell extracts taken from test mammalian subjects, preferably human, with antibody to the ion channel or channels under conditions suitable for complex formation. The amount of complex formation may be quantitated by various methods, preferably by photometric means. Antibodies specific for the mutant ion channels will only bind to individuals expressing the said mutant ion channels and not to individuals expressing only wild-type channels (ie normal individuals). This establishes the basis for diagnosing epilepsy.

Once an individual has been diagnosed with epilepsy, effective treatments can be initiated. Treatments can be directed to amend both mutations or may be directed to one mutation. Treatments may include administering a selective modulator of the mutant ion channel or channels or an antagonist to the mutant ion channel or channels such as an antibody or mutant complement as described above. Alternative treatments include the administering of a selective agonist or modulator to the mutant ion channel or channels so as to restore channel function to a normal level or introduction of wild-type ion channels, particularly through gene therapy approaches as described above. Typically, a vector capable of a expressing the appropriate full length ion channel subunit or subunits or a fragment of derivative thereof may be administered.

In an alternative support approach to therapy, a substantially purified ion channel or ion channel subunit polypeptide and a pharmaceutically acceptable carrier may be administered as described above.

In further embodiments, oligonucleotides or longer fragments derived from any of the polynucleotide sequences forming part of the invention described herein may be used as probes in a microarray. The microarray can be used to monitor the expression level of large numbers of genes simultaneously and to identify genetic variants, mutations, and polymorphisms. This information may be used to determine gene function, to understand the genetic basis of a disorder, to diagnose a disorder, and to develop and monitor the activities of therapeutic agents. Microarrays may be prepared, used, and analyzed using methods known in the art. (For example, see Schena et al., 1996; Heller et al., 1997).

According to a further aspect of the present invention there is provided a method of screening for therapeutic agents useful in the treatment of an IGE, wherein two principal molecular defects in the ion channel subunits of a subject are causative of the IGE, comprising introducing a potential therapeutic agent to a model system in which interaction with the two principal molecular defects is possible, and establishing which therapeutic agents overcome or ameliorate the effect of the two principal defects.

The present invention also provides for the production of genetically modified (knock-out, knock-in and transgenic), non-human animal models transformed with DNA molecules engineered so as to encode two mutant ion channel subunits or with two DNA molecules, each encoding a mutant ion channel subunit. In another approach, genetically modified animals may be produced to contain a specific mutation in just a single ion channel subunit. Mating combinations may then be initiated between such animals so as to produce progeny containing combinations of two ion channel mutations. In both of these approaches, the transgenic animals produced effectively mimic combinations of ion channel mutations that cause human IGE cases and hence represent suitable animal models for these cases. These animals are useful for the study of the function of ion channels, to study the mechanisms by which mutations in two ion channel subunits interact to give rise to epilepsy and the effects of these mutations on brain development, for the screening of candidate pharmaceutical compounds, for the creation of explanted mammalian cell cultures which express mutant ion, channels or combinations of mutant ion channels and for the evaluation of potential therapeutic interventions.

Animal species which are suitable for use in the animal models of the present invention include, but are not limited to, rats, mice, hamsters, guinea pigs, rabbits, dogs, cats, goats, sheep, pigs, and non-human primates such as monkeys and chimpanzees. For initial studies, genetically modified mice and rats are highly desirable due to their relative ease of maintenance and shorter life spans. For certain studies, transgenic yeast or invertebrates may be suitable and preferred because they allow for rapid screening and provide for much easier handling. For longer term studies, non-human primates may be desired due to their similarity with humans.

To create an animal model for a mutated ion channel, or one incorporating a combination of mutations, several methods can be employed. These include but are not limited to generation of a specific mutation in a homologous animal gene, insertion of a wild type human gene and/or a humanized animal gene by homologous recombination, insertion of a mutant (single or multiple) human gene as genomic or minigene cDNA constructs using wild type or mutant or artificial promoter elements or insertion of artificially modified fragments of the endogenous gene by homologous recombination. The modifications include insertion of mutant stop codons, the deletion of DNA sequences, or the inclusion of recombination elements (lox p sites) recognized by enzymes such as Cre recombinase.

To create a transgenic mouse, which is preferred, a mutant version of a particular ion channel subunit or combination of subunits can be inserted into a mouse germs line using standard techniques of oocyte microinjection or transfection or microinjection into embryonic stem cells. Alternatively, if it is desired to inactivate or replace an endogenous ion channel subunit gene, homologous recombination using embryonic stem cells may be applied.

For oocyte injection, one or more copies of the mutant ion channel subunit gene, or combinations thereof, can be inserted into the pronucleus of a just-fertilized mouse oocyte. This oocyte is then reimplanted into a pseudo-pregnant foster mother. The liveborn mice can then be screened for integrants using analysis of tail DNA or DNA from other tissues for the presence of the particular human subunit gene sequence. The transgene can be either a complete genomic sequence injected as a YAC, BAC, PAC or other chromosome DNA fragment, a complete cDNA with either the natural promoter or a heterologous promoter, or a minigene containing all of the coding region and other elements found to be necessary for optimum expression.

While not wishing to be bound by theory, it is believed that two principal molecular defects are causative of the idiopathic generalised epilepsies. In this context there may be interaction between the molecular defects, for example, through the effects of the molecular defects on signaling pathways, which are common for many ion channels.

According to a further aspect of the present invention, neurological material obtained from animal models generated as a result of the identification of specific ion channel subunit human mutations, particularly those disclosed in the present invention, can be used in microarray experiments. These experiments can be conducted to identify the level of expression of particular ion channel subunits or any cDNA clones from whole-brain libraries in epileptic brain tissue as opposed to normal control brain tissue. Variations in the expression level of genes, including ion channel subunits, between the two tissues indicates their involvement in the epileptic process either as a cause or consequence of the original ion channel mutation or mutations present in the animal model. Microarrays may be prepared, used, and analyzed using methods known in the art. (For example, see Schena et al., 1996; Heller et al., 1997).

According to still another aspect of the invention there is provided the use of genetically modified non-human animals as described above for the screening of candidate pharmaceutical compounds.

It will be clearly understood that, although a number of prior art publications are referred to herein, this reference does not constitute an admission that any of these documents forms part of the common general knowledge in the art, in Australia or in any other country.

Throughout this specification and the claims, the words “comprise”, “comprises” and “comprising” are used in a non-exclusive sense, except where the context requires otherwise.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

### BRIEF DESCRIPTION OF THE DRAWINGS

Preferred forms of the invention will now be described, by way of example only, with reference to the following examples and the accompanying drawings, in which:

FIG. 1 is a chart comparing clinical observations with the digenic hypothesis. The left panel shows clinical observations of classical idiopathic generalized epilepsy (IGE) sub-syndromes in various populations. The right panel presents hypothetical explanations of these observations using the digenic model. It is assumed for this illustration that there are five major gene loci for these epilepsies. Mutations in one allele at any two loci can cause IGE, as can mutations in both alleles at a single locus (inbred populations). In this illustration, individuals with similar sub-syndromes in different families have mutations in at least one shared locus. Individuals with a single mutated allele may have febrile seizures (not illustrated);

FIG. 2 illustrates ion channel subunit stoichiometry and the digenic model. A typical channel may have five subunits of three different types (left). In outbred populations idiopathic generalized epilepsies may be due to mutations in two different subunit genes. Because only one allele of each subunit gene is abnormal, half the expressed subunits will have the mutation (middle). In inbred populations, both alleles of a single subunit gene will be affected, so all expressed subunits will be mutated (right);

FIG. 3 represents the location of mutations identified in the ion channel subunits constituting the sodium channel. These examples include both novel and previously identified mutations;

FIG. 4 provides examples of pedigrees where mutation profiles for individuals constituting the pedigree have begun to be determined. These examples have been used to illustrate how the digenic model gives rise to idiopathic generalised epilepsy

### MODES FOR PERFORMING THE INVENTION

Potassium channels are the most diverse class of ion channel. The C. elegans genome encodes about 80 different potassium channel genes and there are probably more in mammals. About ten potassium channel genes are known to be mutated in human disease and include four members of the KCNQ gene sub-family of potassium channels. KCNQ proteins have six transmembrane domains, a single P-loop that forms the selectivity filter of the pore, a positively charged fourth transmembrane domain that probably acts as a voltage sensor and intracellular amino and carboxy termini. The C terminus is long and contains a conserved “A domain” followed by a short stretch thought to be involved in subunit assembly.

Four KCNQ subunits are thought to combine to form a functional potassium channel. All five known KCNQ proteins can form homomeric channels in vitro'and the formation of heteromers appears to be restricted to certain combinations.

Sodium (the alpha subunit) and calcium channels are thought to have evolved from the potassium channel subunit, and they each consist of four domains covalently linked as the one molecule, each domain being equivalent to one of the subunits that associate to form the potassium channel. Each of the four domains of the sodium and calcium channels are comprised of six transmembrane segments.

Voltage-gated sodium channels are required to generate the electrical excitation in neurones, heart and skeletal muscle fibres, which express tissue specific isoforms. Sodium channels are heteromers of a pore forming alpha subunit and a modulatory beta-1 subunit, with an additional beta-2 subunit in neuronal channels. Ten genes encoding sodium channel alpha subunits and 3 genes encoding different beta subunits have so far been identified. The beta subunits of the sodium channels do not associate with the alpha subunits to form any part of the pore, they do however affect the way the alpha pore forming subunit functions.

As with sodium channels, calcium channels consist of a single pore forming alpha subunit, of which at least six types have been identified to date, and several accessory subunits including four beta, one gamma and one alpha2-delta gene. Many of these subunits also encode multiple splice variants adding to the diversity of receptor subunits of this family of ion channels.

The ion channels in the nAChR/GABA super family show a theoretical pentameric channel.

Gamma-Aminobutyric acid (GABA) is the most abundant inhibitory neurotransmitter in the central nervous system. GABA-ergic inhibition is mediated by two major classes of receptors, type A and type B. Type B receptors are members of the class of receptors coupled to G-proteins and mediate a variety of inhibitory effects via secondary messenger cascades. Class A receptors are ligand-gated, chloride channels that mediate rapid inhibition.

The GABA_{A} channel has 16 separate, but related, genes encoding subunits. These are grouped on the basis of sequence identity into alpha, beta, gamma, delta, epsilon, theta and pi subunits. There are six alpha subunits (α1-α6), three beta subunits (β1-β3) and three gamma subunits (γ1-γ3). Each GABA_{A} receptor comprises five subunits which may, at least in theory, be selected from any of these subunits.

Neuronal nicotinic acetylcholine receptors (nAChRs) consist of heterologous pentamers comprising various combinations of alpha subunits or alpha and beta subunits (α2-α9; β2-β4). The alpha subunits are characterised by adjacent cysteine residues at amino acid positions 192 and 193, and the beta subunits by the lack of these cysteine residues. They are ligand-gated ion channels differentially expressed throughout the brain to form physiologically and pharmacologically distinct receptors hypothesised to mediate fast, excitatory transmission between neurons of the central nervous system or to modulate neurotransmission from their presynaptic position.

In chicken and rat, the predominant nAChR subtype is composed of alpha-4 and beta-2 subunits. The transmembrane 2 (M2) segments of the subunits are arranged as alpha helices and contribute to the walls of the neurotransmitter-gated ion channel. The alpha helices appear to be kinked and orientated in such a way that the side chains of the highly conserved M2-leucine residues project inwards when the channel is closed. ACh is thought to cause a conformational change by altering the association of the amino acid residues of M2. The opening of the channel seems to be due to rotations of the gate forming side chains of the amino acid residues; the conserved polar serines and threonines may form the critical gate in the open channel.

### EXAMPLE 1

### Identification of Mutations in Ion Channels Towards Validation of the Digenic Model

The present invention arises from a new genetic model for the idiopathic generalised epilepsies (IGEs) in which it is postulated that IGEs are due to the combination of two mutations in ion channels, in particular, due to separate mutations in each of two subunits of the multiple subunits of the same or different ion channels. Previous studies, including those previously incorporated here-in by reference (Wallace et al., 1998; Australian provisional patents PR4922 and PR2203; Wallace et al., 2001b; Australian patent AU-B-56247/96; Steinlein et al., 1995; PCT/AU01/00541; Phillips et al., 2001; PCT/AU01/00729; Wallace et al., 2001a) have identified mutations in a number of ion channel subunits associated with epilepsy. These include ion channel subunits of voltage-gated (eg SCN1A, SCN1B, KCNQ2, KCNQ3) or ligand-gated (eg CHRNA4, CHRNB2, GABRG2, GABRD) types. To identify further mutations in ion channel genes, particularly those related to the proposed model of idiopathic generalised epilepsy, subunits which comprise the ion channels were screened for molecular defects in IGE patients.

Human genomic sequence available from the Human Genome Project was used to characterize the genomic organisation for each subunit gene. Each gene was subsequently screened for sequence changes using single strand conformation polymorphism (SSCP) analysis in a large sample of epileptics with common sporadic IGE subtypes eg juvenile myoclonic epilepsy (JME), childhood absence epilepsy (CAE), juvenile absence epilepsy (JAE) and epilepsy with generalized tonic-clonic seizures (TCS). Clinical observations can then be compared to the molecular defects characterized in order to establish the combinations of mutant subunits involved in the various disease states, and therefore to provide validated drug targets for each of these disease states. This will provide a basis for novel drug treatments directed at the genetic defects present in each patient.

The coding sequence for each of the ion channel subunits was aligned with human genomic sequence present in available databases at the National Centre for Biotechnology Information (NCBI). The BLASTN algorithm was typically used for sequence alignment and resulted in the genomic organisation (intron-exon structure) of each gene being determined. Where genomic sequence for an ion channel subunit was not available, BACs or PACs containing the relevant ion channel subunit were identified through screening of high density filters containing these clones and were subsequently sequenced.

Availability of entire genomic sequence for each ion channel subunit facilitated the design of intronic primers spanning each exon. These primers were used for both high throughput SSCP screening and direct DNA sequencing.

### EXAMPLE 2

### Sample Preparation for SSCP Screening

A large collection of individuals affected with epilepsy have undergone careful clinical phenotyping and additional data regarding their family history has been collated. Informed consent was obtained from each individual for blood collection and its use in subsequent experimental procedures. Clinical phenotypes incorporated classical IGE cases as well as GEFS⁺ and febrile seizure cases.

DNA was extracted from collected blood using the QIAamp DNA Blood Maxi kit (Qiagen) according to manufacturers specifications or through procedures adapted from Wyman and White (1980). Stock DNA samples were kept at a concentration of 1 ug/ul.

In preparation for SSCP analysis, samples to be screened were formatted into 96-well plates at a concentration of 30 ng/ul. These master plates were subsequently used to prepare exon specific PCR reactions in the 96-well format.

### EXAMPLE 3

### Identification of Sequence Alterations in Ion Channel Genes

SSCP analysis of specific ion channel exons followed by sequencing of SSCP bandshifts was performed on individuals constituting the 96-well plates to identify sequence alterations.

Primers used for SSCP were labelled at their 5′ end with HEX and typical PCR reactions were performed in a total volume of 10 μl. All PCR reactions contained 67 mM Tris-HCl (pH 8.8); 16.5 mM (NH₄)₂SO₄; 6.5 μM EDTA; 1.5 mM MgCl₂; 200 μM each DNTP; 10% DMSO; 0.17 mg/ml BSA; 10 mM P-mercaptoethanol; 5 g/ml each primer and 100 U/ml Taq DNA polymerase. PCR reactions were performed using 10 cycles of 94° C. for 30 seconds, 60° C. for 30 seconds, and 72° C. for 30 seconds followed by 25 cycles of 94° C. for 30 seconds, 55° C. for 30 seconds, and 72° C. for 30 seconds. A final extension reaction for 10 minutes at 72° C. followed.

Twenty μl of loading dye comprising 50% (v/v) formamide, 12.5 mM EDTA and 0.02% (w/v) bromophenol blue were added to completed reactions which were subsequently run on non-denaturing 4% polyacrylamide gels with a cross-linking ratio of 35:1 (acrylamide:bis-acrylamide) and containing 2% glycerol. Gel thickness was 100 μm, width 168 mm and length 160 mm. Gels were run at 1200 volts and approximately 20 mA, at 22° C. and analysed on the GelScan 2000 system (Corbett Research, Australia) according to manufacturers specifications.

PCR products showing a conformational change were subsequently sequenced. This first involved re-amplification of the amplicon from the relevant individual (primers used in this instance did not contain 5′ HEX labels) followed by purification of the PCR amplified templates for sequencing using QiaQuick PCR preps (Qiagen) based on manufacturers procedures. The primers used to sequence the purified amplicons were identical to those used for the initial amplification step. For each sequencing reaction, 25 ng of primer and 100 ng of purified PCR template were used. The BigDye sequencing kit (ABI) was used for all sequencing reactions according to the manufacturers specifications. The products were run on an ABI 377 Sequencer and analysed using the EditView program.

Tables 1A and 1B show the sequence changes identified in some of the ion channel subunits to date. These changes, along with previously identified polymorphisms or mutations as well as changes not yet identified can be used to test the digenic hypothesis

### EXAMPLE 4

### Digenic Model Examples

The digenic model may be validated through a parametric analysis of large families in which two abnormal alleles co-segregate by chance to identify mutations which act co-operatively to give an epilepsy phenotype. It is envisaged that the strategy of careful clinical phenotyping in these large families, together with a linkage analysis based on the digenic hypothesis will allow identification of the mutations in ion channels associated with IGEs. If molecular genetic studies in IGE are successful using the digenic hypothesis, such an approach might serve as a model for other disorders with complex inheritance.

The digenic hypothesis predicts that the closer the genetic relationship between affected individuals, the more similar the sub-syndromes, consistent with published data (Italian League Against Epilepsy Genetic Collaborative Group, 1993). This is because more distant relatives are less likely to share the same combinations of mutated subunits.

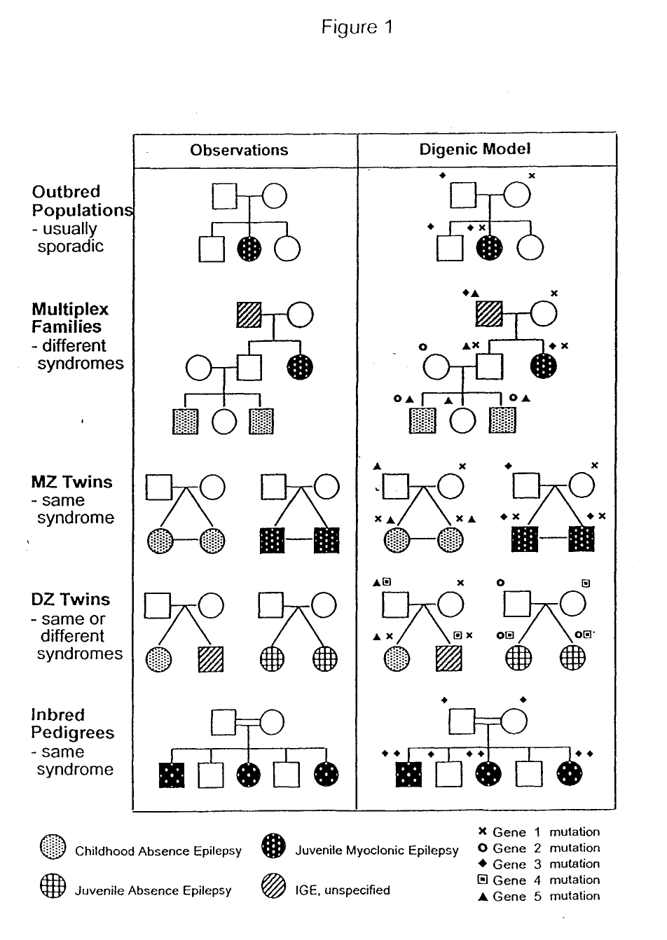

Identical twins have the same pair of mutated subunits and the same minor alleles so the sub-syndromes are identical. Affected sib-pairs, including dizygous twins, with the same sub-syndrome would also have the same pair of mutated subunits, but differences in minor alleles would lead to less similarity than with monozygous twins. Some sib-pairs and dizygous twins, have quite different sub-syndromes; this would be due to different combinations of mutated subunits, when the parents have more than two mutated alleles between them (FIG. 1, right panel).

A special situation exists in inbred communities that parallels observations on autosomal recessive mouse models. Here the two mutated alleles of the digenic model are the same and thus result in a true autosomal recessive disorder. Because all affected individuals have the same pair of mutated alleles, and a similar genetic background, the phenotypes are very similar (FIG. 1, right panel).

In outbred communities approximately 1% of the population would have IGE genotypes (2 mutated alleles) and 0.3% would clinically express IGE (FIG. 1, right panel). Most of these would have mutations in two different channel subunits. In such communities most cases would appear “sporadic” as the risk to first degree relatives would be less than 10%.

For example, let there be three IGE loci (A,B,C) and let the frequency of abnormal alleles (a*,b*,c*) at each locus be 027 and of normal alleles (a, b, c) be 0.973. Then, the distribution of genotypes aa*, a*a, a*a* and aa at locus A will be 0.0263 (0.027×0.973), 0.0263, 0.0007 and 0.9467 respectively, and similarly for loci B and C. In this population 0.8485 will have no mutated alleles (0.94673), 0.1413 will have one mutated allele (a* or b* or c*; 0.0263×0.94672×6), 0.0098 will have two abnormal alleles (0.0020 two same abnormal alleles, 0.0078, two different abnormal alleles) and 0.00037 will have more than two abnormal alleles. Thus in this population 0.01, or 1%, will have two or more abnormal alleles (IGE genotype), and the total abnormal allele frequency will be 0.08 (3×0.027).

To determine the familial risks and allele patterns in affected pairs, the frequency distribution of population matings and the percentage of children with 2 or more abnormal alleles must be determined. The frequency of matings with no abnormal alleles (0×0) is 0.72 (0.84852), for 1×0 and 0×1 matings 0.24 (2×0.8485×0.1413), for a 1×1 mating 0.020, and for 2×0 and 0×2 matings 0.0166 etc. From this distribution of matings the frequency of children with 2 or more abnormal alleles can be shown to be 0.01. For example, the 0×2 and 2×0 matings contribute 0.0033 of this 0.01 frequency (0.0166 [mating frequency]×0.2 [chance of that mating producing a child with 2 or more abnormal alleles]).

To determine parental risk it can be shown that of children with 2 abnormal alleles (IGE genotype), 0.49 derive from 1×1 matings where no parent is affected, 0.33 derive from a 2×0 and 0×2 matings etc. For the 2×0 and 0×2 matings, half the parents have IGE genotypes and contribute 0.16 (0.33/2) to the parental risk with the total parental risk of an IGE genotype being 0.258. The other matings that contribute to affected parent-child pairs are 2×1, 1×2, 3×0, 0×3 etc.

The sibling risk of an IGE genotype is 0.305. For example 2×0 and 0×2 matings contributed 0.08 to the sibling risk (0.33[fraction of children with 2 abnormal alleles]×0.25[the chance of that mating producing a child with 2 or more abnormal alleles]). Similarly the offspring risk was determined to be 0.248 by mating individuals with 2 abnormal alleles with the general population. Thus at 30% penetrance the risk for IGE phenotype for parents of a proband is 0.077, for siblings 0.091, and for offspring 0.074.

It can be shown that affected sib pairs share the same abnormal allele pair in 85% of cases. This is because of all affected sib pairs 44% derive from 1×1 matings and 23% from 0×2 and 2×0 matings where all affected siblings have the same genotype. In contrast, 24% derive from 1×2 matings and 9% from 3×1 and 2×2 matings etc where affected sibling genotypes sometimes differ.

For affected parent-child pairs, genotypes are identical in only 58%. Of affected parent child pairs, 43% derive from 0×2 matings where gentoypes are identical, whereas 38% derive from 0×3 and 17% from 1×2 where the majority of crosses yield different affected genotypes.

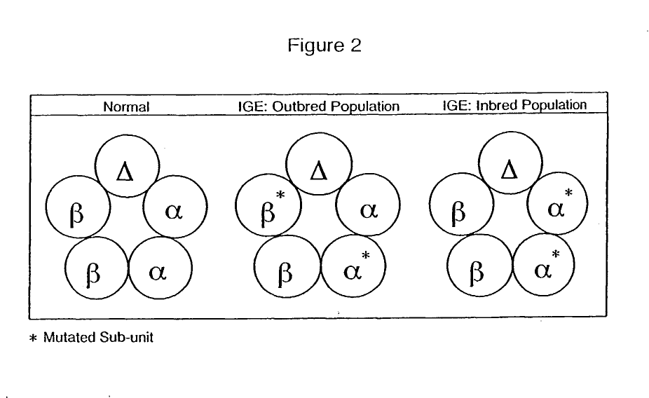

The hypothesis that similar phenotypes can be caused by the combination of mutations in two different subunits (outbred communities), or by the same mutation in two alleles of the same subunit (inbred communities), may seem implausible. However, applying the digenic hypothesis to the theoretical pentameric channel shown in FIG. 2, in outbred communities IGE will be due to subunit combinations such as α*αβ*βΔ, α*αββΔ* or ααβ*βΔ* (mutated subunits indicated by *). In inbred communities α*α*ββΔ or ααβ*β*Δ combinations might cause IGE phenotypes. We assume that the mutations will not cause reduced expression of the alleles and that the altered ion channel excitability, and consequent IGE phenotype, caused by mutations in two different alleles is similar to that caused by the same mutation in both alleles of one subunit.

The relative separate segregation of classical IGE and GEFS⁺ phenotypes is an anecdotal clinical observation of ours (Singh et al., 1999), although the separation is not absolute. The separation is supported by previous family and EEG studies of Doose and colleagues who described “type A” and “type B” liabilities which we may approximate the GEFS⁺ and classical IGE groupings respectively (Doose and Baier, 1987).

The digenic model predicts that affected sib pairs will share the same genes in 85% of cases whereas they will have at least one different allele in the remaining 15%. In contrast, only 58% of parent-child pairs share the same alleles in a 3 locus model. Thus there should be greater similarity of syndromes between sibling pairs than parent-child pairs. This would be most objectively measured by age of onset and seizure types.

Estimates for the risk of febrile seizures or IGE in relatives vary. The estimates range from 5%-10% for siblings, 4%-6% for offspring, 3%-6% for parents, and 2-3% for grandparents. Underestimation may occur because IGE manifest in youth, and parents and particularly grandparents may be unaware of seizures in themselves in younger years. This is particularly true where there was stigma associated with epilepsy and where the epilepsy may have been mild and unrecognized. Underestimation of sibling and offspring risks occurs when unaffected young children are counted, some of whom will develop IGE in adolescence. Overestimation may occur with misdiagnosis of seizures or inclusion of seizures unrelated to IGE (e.g. due to trauma or tumors)

In autosomal dominant models the risk to affected relatives reduces proportionally (50% for first degree relatives, 25% for second degree etc). For all oligogenic or polygenic models the risk decreases more quickly. For a digenic model with three loci, the risks are 9.1% for siblings, 7.4% for offspring, 7.7% for parents. Rigorous measurement of the familial recurrence rates, with careful phenotyping and age-corrected risk estimates could be compared with the predictions from the digenic model, and it is proposed to do this.

There is a small amount of information on IGE families regarding haplotype distribution. For example, there is some evidence for a locus on 8q as determined by parametric linkage in a single family (Fong et al., 1998) and by non-parametric analysis in multiple small families (Zara et al., 1995). Interestingly, in the latter study the 8q haplotype not infrequently came from the unaffected parent. This would be quite compatible with the digenic model and evaluation of other data sets in this manner could be used to test the hypothesis, and it is proposed to do this.

Following the analysis of one large family with epilepsy where the two main phenotypes were childhood absence epilepsy (CAE) and febrile seizures (FS), the inheritance of FS was found to be autosomal dominant and the penetrance 75%. However the inheritance of CAE in this family was not simple Mendelian, but suggestive of complex inheritance with the involvement of more than one gene. The power of this large family was used to explore the complex genetics of CAE further.

Linkage analysis on this family in which individuals with CAE, FS and FS+were deemed affected led to the detection of linkage on chromosome 5q and identification of a mutation in the GABRG2 gene (R43Q) which is localised to this region (Wallace et al., 2001a; PCT/AU01/00729). All 10 tested individuals with FS alone in this family had this mutation and 7 CAE affected individuals in this family also had the mutation. To test the digenic model of IGEs in the CAE affected individuals, the whole genome screen of this family was reanalysed with only individuals with CAE considered affected. Linkage analysis was performed using FASTLINK v40, two-point lod scores were calculated assuming 50% penetrance and a 2% phenocopy rate and individuals with FS or FS+were coded as unknown. Markers producing a lod score greater than 1 were reanalysed without a phenocopy rate and at the observed penetrance for CAE in this family (30%). Results from the analysis revealed significant linkage to chromosome 14q22-q23 (lod 3.4). This provides strong evidence for a second locus segregating with CAE affected individuals in this family. While the GABRG2 mutation is sufficient to cause FS, the CAE phenotype is thought to be due to both the GABRG2 mutation and a mutation occurring in a gene mapping to the 14q locus, as proposed by the digenic model.

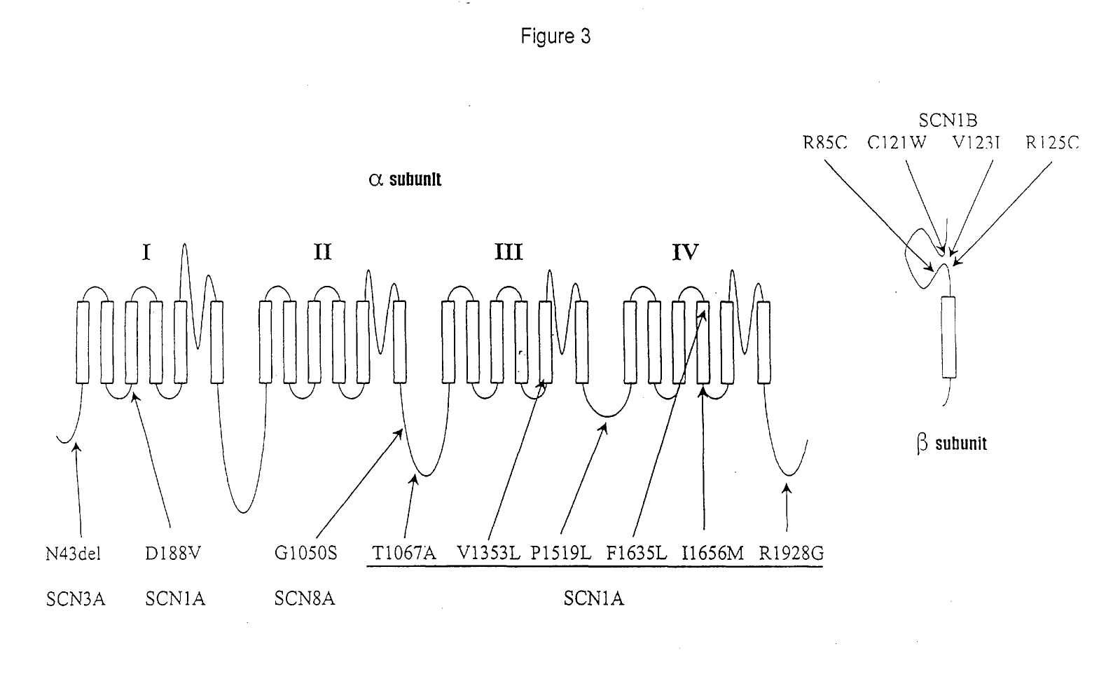

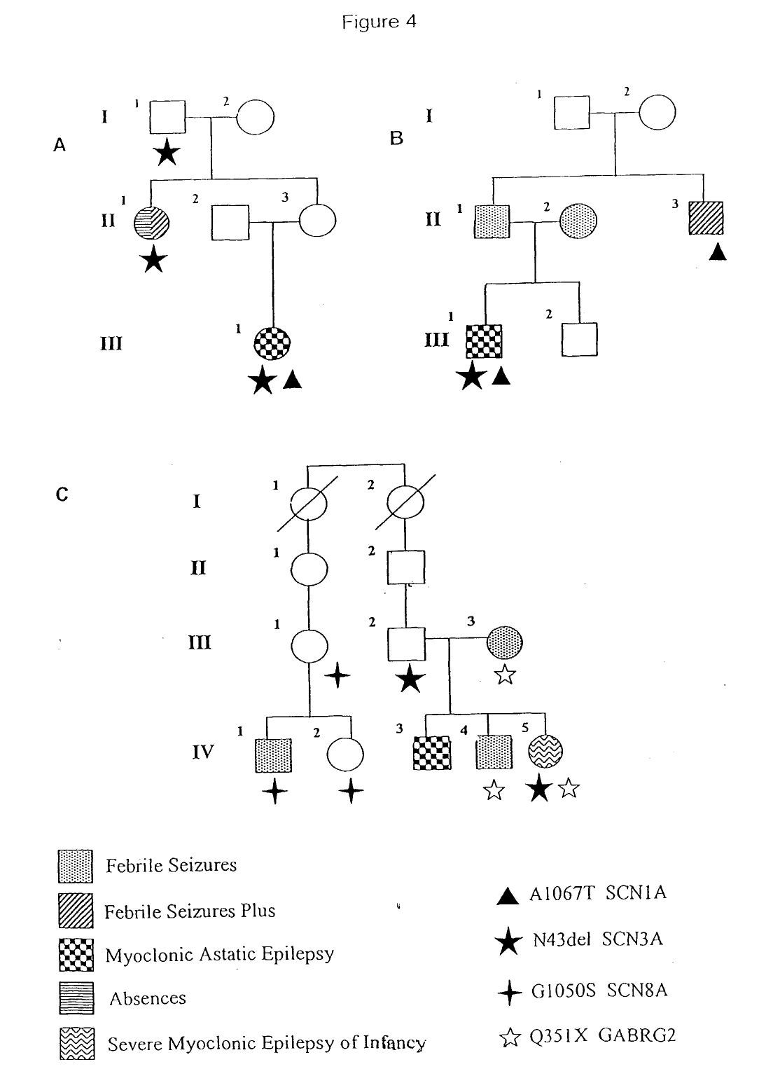

For the application of the digenic model to sporadic cases of IGE and affected individuals belonging to smaller families in which genotyping and linkage analysis is not a feasible approach to disease gene identification, direct mutation analysis of ion channel genes in these individuals has been carried out as described above. In Tables 1A and 1B there is provided an indication of genetic alterations so far identified through mutation analysis screening of these individuals. FIG. 3 provides an example to indicate where some of these mutations have occurred with respect to the sodium channel genes. To further test the digenic hypothesis, mutation profiles are starting to accumulate for a number of subunits expressed in the same individuals. FIG. 4 provides results from some of these profiles.

FIG. 4A shows a 3 generation family in which individual III-1 has myoclonic astatic epilepsy and contains a N43del mutation in the SCN3A gene as well as an A1067T mutation in the SCN1A gene. Individual I-1 also has the SCN3A mutation but alone this mutation is not sufficient to cause epilepsy in this individual. The SCN3A mutation has likely been inherited from the grandfather through the mother, while the SCN1A mutation is likely to arise from the father. Both parents are unaffected but have yet to be screened for the presence of the mutations in these subunits. Individual II-1 is likely to contain an as yet unidentified ion channel subunit mutation acting in co-operation with the SCN3A mutation already identified in this individual.

FIG. 4B is another 3 generation family in which individual III-1 has myoclonic astatic epilepsy due to a combination of the same SCN3A and SCN1A mutations as above. However, in this family both parents have febrile seizures most likely due to the presence of just one of the mutations in each parent, as proposed by the model. This is in contrast to individuals II-2 and II-3 in FIG. 4A who also contain one of the mutations in these genes each. These individuals are phenotypically normal most likely due to incomplete penetrance of these mutations in each case.

FIG. 4C shows a larger multi-generation family in which individual IV-5 has a mutation in both the SCN3A and GABRG2 subunits. In combination, these give rise to severe myoclonic epilepsy of infancy but alone either cause febrile seizures (GABRG2 mutation in III-3 and IV-4) or are without an effect (SCN3A mutation in III-2) as proposed by the model.

These examples therefore illustrate the digenic model as determined from mutation analysis studies of ion channel subunits in affected individuals.

### EXAMPLE 5

### Analysis of Receptors and Receptor Subunits

The following methods are used to determine the structure and function of the ion channels and ion channel subunits.

Molecular Biological Studies

The ability of any one of the ion channels that form part of this invention to bind known and unknown proteins as a whole or through individual subunits can be examined. Procedures such as the yeast two-hybrid system are used to discover and identify any functional partners. The principle behind the yeast two-hybrid procedure is that many eukaryotic transcriptional activators, including those in yeast, consist of two discrete modular domains. The first is a DNA-binding domain that binds to a specific promoter sequence and the second is an activation domain that directs the RNA polymerase II complex to transcribe the gene downstream of the DNA binding site. Both domains are required for transcriptional activation as neither domain can activate transcription on its own. In the yeast two-hybrid procedure, the gene of interest or parts thereof (BAIT), is cloned in such a way that it is expressed as a fusion to a peptide that has a DNA binding domain. A second gene, or number of genes, such as those from a cDNA library (TARGET), is cloned so that it is expressed as a fusion to an activation domain. Interaction of the protein of interest with its binding partner brings the DNA-binding peptide together with the activation domain and initiates transcription of the reporter genes. The first reporter gene will select for yeast cells that contain interacting proteins, (this reporter is usually a nutritional gene required for growth on selective media). The second reporter is used for confirmation and while being expressed in response to interacting proteins it is usually not required for growth.

Ion channel interacting genes may also be targets for mutation and when expressed in combination with another ion channel mutation or ion channel interacting gene mutation (as proposed by the digenic model of this invention) may give rise to epilepsy.

The nature of the ion channel interacting genes and proteins can also be studied such that these partners can also be targets for drug discovery.

Structural Studies

Ion channel recombinant proteins can be produced in bacterial, yeast, insect and/or mammalian cells and used in crystallographical and NMR studies. Together with molecular modelling of the protein, structure-driven drug design can be facilitated.

### EXAMPLE 6

### Generation of Polyclonal Antibodies Specific for Ion Channels or Ion Channel Subunits

Following the confirmation that two ion channel mutations act synergistically to give rise to IGE, antibodies can be made to selectively bind and distinguish mutant from normal proteins. Antibodies specific for mutagenised epitopes are especially useful in cell culture assays to screen for cells which have been treated with pharmaceutical agents to evaluate the therapeutic potential of the agent.

To prepare polyclonal antibodies, short peptides can be designed homologous to a particular ion channel subunit amino acid sequence. Such peptides are typically 10 to 15 amino acids in length. These peptides should be designed in regions of least homology to other ion channel subunits and should also have poor homology to the mouse orthologue to avoid cross species interactions in further down-stream experiments such as monoclonal antibody production. Synthetic peptides can then be conjugated to biotin (Sulfo-NHS-LC Biotin) using standard protocols supplied with commercially available kits such as the PIERCE™ kit (PIERCE). Biotinylated peptides are subsequently complexed with avidin in solution and for each peptide complex, 2 rabbits are immunized with 4 doses of antigen (200 μg per dose) in intervals of three weeks between doses. The initial dose is mixed with Freund's Complete adjuvant while subsequent doses are combined with Freund's Immuno-adjuvant. After completion of the immunization, rabbits are test bled and reactivity of sera is assayed by dot blot with serial dilutions of the original peptides. If rabbits show significant reactivity compared with pre-immune sera, they are then sacrificed and the blood collected such that immune sera can be separated for further experiments.

This procedure is repeated to generate antibodies against wild-type forms of ion channel subunits. These antibodies, in conjunction with antibodies to mutant ion channel subunits, are used to detect the presence and the relative level of the mutant forms in various tissues.

### EXAMPLE 7

### Generation of Monoclonal Antibodies Specific for Ion Channels or Ion Channel Subunits

Monoclonal antibodies can be prepared in the following manner. Immunogen comprising an intact ion channel subunit protein or ion channel subunit peptides (wild type or mutant) is injected in Freund's adjuvant into mice with each mouse receiving four injections of 10 to 100 ug of immunogen. After the fourth injection blood samples taken from the mice are examined for the presence of antibody to the immunogen. Immune mice are sacrificed, their spleens removed and single cell suspensions are prepared (Harlow and Lane, 1988). The spleen cells serve as a source of lymphocytes, which are then fused with a permanently growing myeloma partner cell (Kohler and Milstein, 1975). Cells are plated at a density of 2×10⁵ cells/well in 96 well plates and individual wells are examined for growth. These wells are then tested for the presence of ion channel subunit specific antibodies by ELISA or RIA using wild type or mutant subunit target protein. Cells in positive wells are expanded and subcloned to establish and confirm monoclonality. Clones with the desired specificity are expanded and grown as ascites in mice followed by purification using affinity chromatography using Protein A Sepharose, ion-exchange chromatography or variations and combinations of these techniques.

### INDUSTRIAL APPLICABILITY

The present invention is useful in the identification and characterisation of ion channel genes responsible for IGE and in the subsequent diagnosis and treatment of IGE.

### REFERENCES

References cited herein are listed on the following pages, and are incorporated herein by this reference.

Andermann, E. (1982). In: Genetic basis of the epilepsies. Anderson, V E. Hauser, W A. Penry, J K. and Singh, C F. (Editors). New York, Raven Press. 355-374.

Bell, J I. and Lathrop, M. (1996). Nature Genet. 13: 377-378.

Berkovic, S F. Andermann, F. Andermann, E. and Gloor, P. (1987). Neurology 37: 993-1000.

Berkovic, S F. Reutens, D C. Andermann, E. and Andermann, F. (1994). In: Epileptic seizures and syndromes. Wolf, P. (Editor) London: John Libbey. 25-37.

Biervert, C. Schroeder, B C. Kubisch, C. Berkovic, S F. Propping, P. Jentsch, T J. and Steinlein, O K. (1998). Science 279: 403-406.

Cavazzuti, G B. Capella, L. and Nalin, A. (1980). Epilepsia 21: 43-55.

Charlier, C. Singh, N A. Ryan, S G. Lewis, T B. Reus, B E. Leach, R J. and Leppect, M. (1998). Nature Genet. 18: 53-55.

Cole, S P. Campling, B G. Atlaw, T. Kozbor, D. and Roder, J C. (1984). Mol. Cell Biochem. 62: 109-120.

Collins, F S. (1995). Nature Genet. 9: 347-350.

Commission on Classification and Terminology of the International League against Epilepsy. (1989). Epilepsia 30: 389-399.

Cote, R J. Morrissey, D M. Houghton, A N. Beattie, E J Jr. Oettgen, H F. and Old, L J. (1983). Proc. Natl. Acad. Sci. USA 80: 2026-2030.

Culver, K. (1996). Gene Therapy: A Primer for Physicians. Second Edition. (Mary Ann Liebert).

Doose, H. and Baier, W K. (1987). Neuropediatrics 18 (Supplement 1): 1-64.

Doose, H. and Baier, W. (1989). Clev. Clin. J. Med. 56 (Supplement): s105-s110.

Escayg, A. MacDonald, B T. Meisler, M H. Baulac, S. Huberfeld, G. An-Gourfinkel, I. Brice, A. LeGuern, E. Moulard, B. Chaigne, D. Buresi, C. and Malafosse, A. (2000). Nature Genet. 24: 343-345.

Fong, G C. Shah, P U. Gee, M N. Serratosa, J M. Castroviejo, I P. Khan, S. Ravat, S H. Mani, J. Huang, Y. Zhao, H Z. Medina, M T. Treiman, L J. Pineda, G. and Delgado-Escueta, A V. (1998). Am. J. Hum. Genet. 63: 1117-1129.

Friedman, T. (1991). In: Therapy for Genetic Diseases. Friedman, T. (Editor). Oxford University Press. pp 105-121.

Goldman, C K. Soroceanu, L. Smith, N. Gillespie, G Y. Shaw, W. Burgess, S. Bilbao, G. and Curiel, D T. (1997). Nature Biotechnology 15: 462-466.

Greenberg, D A. Delagado-Escueta, A V. Maldonado, H M. and Widellitz, H. (1988). Genet Epidem. 5: 81-94.

Greenberg, D A. Delgado-Escueta, A V. Widelitz, H. Sparkes, R S. Treiman, L. Maldonado, H M. Park, M S. and Terasaki, P I. (1988). Am. J. Med. Genet. 31: 185-192.

Greenberg, D A. Durner, M. and Delgado-Escueto, A V. (1992). Neurology 42 (Supplement 5): 56-62.

Harlow, E. and Lane, D. (1988). Antibodies: A Laboratory Manual (Cold Spring Harbor Laboratory, Cold Spring Harbor, N.Y.).

Hauser, W A. Annegers, J F. and Kurland, L T. (1993). Epilepsia 34: 453-468.

Heller, R A. Schena, M. Chai, A. Shalon, D. Bedilion, T. Gilmore, J. Woolley, D E. and Davis R W. (1997). Proc. Natl. Acad. Sci. USA 94: 2150-2155.

Huse, W D. Sastry, L. Iverson, S A. Kang, A S. Alting-Mees, M. Burton, D R. Benkovic, S J. and Lerner, R A. (1989). Science 246: 1275-1281.

Italian League Against Epilepsy Genetic Collaborative Group. (1993). Epilepsia 34: 819-26.

Janz, D. Beck-Mannagetta, G. and Sander, T. (1992). Neurology 42 (Supplement 5): 48-55.

Kohler, G. and Milstein, C. (1975). Nature 256: 495-497.

Kozbor, D. Abramow-Newerly, W. Tripputi, P. Cole, S P. Weibel, J. Roder, J C. and Croce, C M. (1985). J. Immunol. Methods 81:31-42.

Lernmark, A. and Ott, J. (1998). Nature Genet. 19: 213-214.

Okubo, Y. Matsuura, M. Asai, T. Asai, K. Kato, M. Kojima, T. and Toru, M. (1994). Epilepsia 35: 832-841.

Orlandi, R. Gussow, D H. Jones, P T. and Winter, G. (1989). Proc. Natl. Acad. Sci. USA 86: 3833-3837.

Panayiotopoulos, C P. and Obeid, T. (1989). Ann. Neurol. 25: 440-443.

Phillips, H A. Favre, I. Kirkpatrick, M. Zuberi, S M. Goudie, D. Heron, S E. Scheffer, I E. Sutherland, G R. Berkovic, S F. Bertrand, D. and Mulley, J C. (2001). Am. J. Hum. Genet. 68: 225-231.

Reutens, D C. and Berkovic, S F. (1995). Neurology 45: 1469-1476.

Risch, N. and Botstein, D. (1996). Nature Genet. 12: 351-353.

Roger, J. Bureau, M. Dravet, C. Dreifuss, F E. Perret, A. and Wolf, P. (1992). Epileptic syndromes in infancy, childhood and adolescence. 2nd Edition. London, John Libbey.

Scharf, K D. Materna, T. Treuter, E. and Nover, L. (1994). Results Probl. Cell Differ. 20: 125-162.

Scheffer, I E. and Berkovic, S F. (1997). Brain 120: 479-90.

Schena, M. Shalon, D. Heller, R. Chai, A. Brown, P O. and Davis, R W. (1996). Proc. Natl. Acad. Sci. USA 93: 10614-10619.

Singh, N A. Charlie, C. Stauffer, D. DuPont, B R. Leach, R J. Melis, R. Ronen, G M. Bjerre, I. Quattlebaum, T. Murphy, J V. McHarg, M L. Gagnon, D. Rosales, T O. Peiffer, A. Anderson, V E. and Leppert, M. (1998). Nature Genet. 18: 25-29.

Singh, R. Scheffer, I E. Crossland, K. and Berkovic, S F. (1999). Ann. Neurol. 45: 75-81.

Steinlein, O K. Mulley, J C. Propping, P. Wallace, R H. Phillips, H A. Sutherland, G R. Scheffer, I E. and Berkovic, S F. (1995). Nature Genet. 11: 201-203.

Todd, J A. (1999). Lancet 354 (Supplement 1): 15-16.

Wallace, R H. Marini, C. Petrou, S. Harkin, L A. Bowser, D N. Panchal, R G. Williams, D A. Sutherland, G R. Mulley, J C. Scheffer, I E. and Berkovic, S F. (2001a). Nature Genet. 28: 49-52.

Wallace, R H. Scheffer, I E. Barnett, S. Richards, M. Dibbens, L. Desai, R R. Lerman-Sagie, T. Lev, D. Mazarib, A. Brand, N. Ben-Zeev, B. Goikhman, I. Singh, R. Kremmidiotis, G. Gardner, A. Sutherland, G R. George, A L Jr. Mulley, J C. and Berkovic, S F. (2001b). Am. J. Hum. Genet. 68: 859-865.

Wallace, R H. Wang, D W. Singh, R. Scheffer, I. George, A. Phillips, H. Saar, K. Reis, A. Johnson, E. Sutherland, G. Berkovic, S. and Mulley, J. (1998). Nature Genet. 19: 366-370.

Winter, G. and Milstein, C. (1991). Nature 349: 293-299.

Wyman, A R. and White, R. (1980). Proc. Natl. Acad. Sci. 77: 6754-6758.

Zara, F. Bianchi, A. Avanzini, G. Di Donato, S. Castellotti, B. Patel, P I. and Pandolfo, M. (1995). Hum. Mol. Genet. 4: 1201-1207.

Zara, F. Gennaro, E. Stabile, M. Carbone, I. Malacarne, M. Majello, L. Santangelo, R. de Falco, F A. and Bricarelli, F D. (2000). Am. J. Hum. Genet. 66: 1552-1557.

1. A method for identifying the molecular defects responsible for the idiopathic generalised epilepsies (IGE), comprising the steps of:
1) providing sequence information for ion channel subunits;
2) screening a nucleic acid or peptide isolated from a patient affected by an IGE for molecular defects in the ion channel subunits in order to identify two principal defects associated with the IGE; and
3) correlating the two principal molecular defects identified with clinical observations in order to establish the combination of mutant subunits involved in the IGE.

2. A method as claimed in claim 1, further comprising the step of repeating said screening step for a plurality of patients with different IGEs in order to establish the combinations of mutant subunits involved in the various IGEs.

3. A method as claimed in claim 1 or claim 2, further comprising the step of establishing whether two principal defects can be associated with a selected IGE.

4. A method as claimed in any one of claims 1 to 3, further comprising the step of identifying further molecular defects in genes of smaller effect.

5. A method as claimed in any one of claims 1 to 4 wherein the molecular defects are selected from the group consisting of molecular defects in voltage-gated ion channel subunits and molecular defects in ligand-gated ion channel subunits.

6. A method as claimed in claim 5 wherein the voltage-gated ion channel subunits are sodium channel subunits or potassium channel subunits and the ligand-gated ion channel subunits are Gamma-Aminobutyric acid receptor subunits or nicotinic acetylcholine receptor subunits.

7. A method as claimed in claim 6 wherein the voltage-gated ion channel subunits are selected from the group consisting of SCN1A, SCN3A, SCN8A, SCN1B, KCNQ2, KCNQ3 and KCNQ5 and the ligand-gated ion channels are selected from the group consisting of GABRA1, GABRA2, GABRA4, GABRA5, GABRB1, GABRB3, GABRD, GABRG2 and GABRG3.

8. A method as claimed in claim 7 wherein the molecular defects are selected from the group consisting of:

| | | | |
|---|---|---|---|
| SCN1A | Exon 4 | c563A→T | D188V |
| SCN1A | Exon 21 | c4057G→4C | V1353L |
| SCN1A | Exon 24 | c4556C→T | P1519L |
| SCN1A | Exon 26 | c4905C→G | F1635L |
| SCN1A | Exon 26 | c4968C→G | I1656M |
| SCN8A | Exon 14 | c3148G→A | G1050S |
| SCNiB | Exon 3 | c253C→T | R85C |
| SCN1B | Exon 3 | c363C→G | C121W |
| SCN1B | Exon 3 | c367G→A | V123I |
| SCN1B | Exon 3 | c373C→T | R125C |
| SCN1A | Exon 16 | c3199A→G | T1067A |
| SCN1A | Exon 26 | c5782C→G | R1928G |
| SCN3A | Exon 1 | c127-129delAAT | N43del |
| SCNlA | Exon 15 | c2889T→C | — |
| SCN1A | Exon 14 | c2522C→G | — |
| SCN8A | Intron 15 | IVS15 + 20G→A | — |
| KCNQ3 | Exon 15 | c2306C→A | P769H |
| KCNQ2 | Exon 15 | c2255C→A | T752N |
| KCNQ5 | Exon 14 | c1869A→T | — |
| KCNQ2 | Exon 6 | c912C→T | — |
| KCNQ2 | Exon 11 | c1419C→G | — |
| KCNQ2 | Exon 15 | c2154T→A | — |
| KCNQ2 | Exon 15 | c2460G→A | — |
| KCNQ3 | Exon 4 | c660T→C | — |
| KCNQ3 | Exon 4 | c732T→C | — |
| KCNQ3 | Exon 7 | c1071C→G | — |
| KCNQ2 | Intron 11 | IVS11 + 1G→A | — |
| GABRD | Exon 5 | c530A→C | E177A (E129A) |
| GABRD | Exon 6 | c658C→T | R220C (R172C) |
| GABRG2 | Exon 2 | c245G→A | R82Q (R43Q) |
| GABRG2 | Exon 9 | c1168C→T | Q390X (Q351X) |
| CABRD | Exon 6 | c659G→A | R22OH (R172C) |
| GABRAS | Exon 5 | c235A→C | 179L (I48L.) |
| GABRA4 | Exon 1 | c76C→A | L26N (signal peptide) |
| GABRB3 | Exon 6 | c603C→T | — |
| GABRB3 | Exon 7 | c783G→A | — |
| GABRB3 | Exon 8 | c1005C→T | — |
| GABRB3 | Exon 9 | c1293G→A | — |
| GABRA1 | Exon 11 | c1155C→A | — |
| GABRA1 | Exon 11 | c1440A→G | — |
| GABRD | Exon 4 | c405C→T | — |
| GABRD | Exon 4 | c444C→T | — |
| GABRA2 | Exon 7 | c513G→A | — |
| GABRB3 | Exon 1A | c(1A)75C→T | — |
| GABRB1 | Exon 8 | c846A→G | — |
| GABRA1 | Exon 4 | c156T→C | — |
| GABRD | Exon 4 | c330C→T | — |
| GABRD | Exon 4 | c816C→T | — |
| GABRD | Exon 9 | c1104C→T | — |
| GABRG2 | Exon 3 | c315C→T | — |
| GABRG2 | Exon 5 | c588T→C | — |
| GABRA2 | Exon 6 | c396G→A | — |
| GABRA5 | Exon 8 | c606T→C | — |
| GABRA5 | Exon 10 | c975T→C | — |
| GABRG3 | Intron 5 | IVS5 + 20C→T | — |
| GABRG2 | Intron 1 | IVS1 + 12c→T | — |
| GABRG3 | Intron 1 | IVS1 + 11C→T | — |
| GABRB3 | Intron 8 | IVS8 + 15A→G | — |
| GABRD | Intron 1 | IVS1 − 17A→G | — |
| GABRD | Intron 8 | IVS8 − 7C→T | — |
| GABRD | Intron 8 | IVS8 − 14C→T | — |
| GABRB2 | Intron 6 | IVS6 − 11T→C | — |
| GABRA3 | Intron 1 | IVS1 − 21− 22insT | — |
| GABRB3 | Promoter | IVS1A − 43G→A | — |
| CNRNA4 | Exon 5 | c770T→G | I257S |
| CNRNA4 | Exon 5 | c839C→T | S280F |
| CHRNB2 | Exon 5 | c859G→A | V287M |
| CHRNB2 | Exon 5 | c901C→G | L301V |
| CHRNB2 | Exon 5 | c1235G→A | G412D |
| CHRNB2 | Exon 5 | c1191G→C | Q397H |
| CNRNA4 | Exon 1 | c51G→A | — |
| CNRNA4 | Exon 5 | c1629C→T | — |
| CNRNA4 | Exon 5 | c1659G→A | — |
| CHRNB2 | Exon 2 | c109C→T | — |
| CHRNB2 | Exon 5 | c1233G→A | |
| CHRNB2 | Exon 6 | c1482A→G | |
| CHRNA4 | Intron 5 | IVS + 11C→T | |
| CHRNA4 | Intron 5 | IVS5 + 14G→A | |
| CHRNB2 | Intron 5 | IVS5 + 14G→A | |

in which the left-hand column lays out the ion channel in which the molecular defect occurs, the second column identifies the exon or intron of the ion channel gene in which the molecular defect occurs, the third column describes the DNA mutation and, where an amino acid change occurs, the fourth column gives the amino acid change.

9. A method as claimed in claim 8 wherein one principal molecular defect is a N43del mutation in the SCN3A gene.

10. A method as claimed in claim 9 wherein a second principal molecular defect is an A1067T mutation in the SCN1A gene

11. A method as claimed in claim 10 wherein a second principal molecular defect is an R43Q mutation in GABRG2.

12. A method as claimed in any one of claims 1 to 11 wherein genomic DNA is screened for mutations and/or polymorphisms in exons or in regions adjacent an intron/exon boundary.

13. A method as claimed in claim 12 wherein a single strand conformation polymorphism (SSCP) analysis is conducted.

14. A method for establishing the loci of molecular defects of ion channel subunit genes responsible for the idiopathic generalised epilepsies (IGE), comprising the steps of:
1) obtaining a DNA sample from a patient affected with epilepsy;
2) comparing the DNA sample with that from a corresponding wild type DNA sample;
3) identifying those patient DNA samples in which two loci segregate with the disease; and
4) determining the location of the two abnormal loci.

15. A method as claimed in claim 14 wherein a linkage analysis is performed in a large family.

16. A method as claimed in claim 14 or claim 15 further comprising the step of identifying the genes with which the abnormal alleles are associated.

17. A method of diagnosis of an IGE, comprising the steps of:
1) providing a DNA sample from a patient suspected of an IGE;
2) screening the sample for molecular defects in the genes encoding ion channel subunits;
3) establishing the presence or absence of mutations associated with IGE and, where such mutations are present, identifying the two principal mutations associated with the IGE;
4) comparing the identified molecular defects to pre-existing data correlating molecular defects to sub-syndromes of IGE; and
5) identifying the IGE.

18. A method of diagnosis of an IGE, comprising the steps of:
1) providing a protein sample from a patient suspected of an IGE;
2) screening the sample for molecular defects in ion channel subunits;
3) establishing the presence or absence of molecular defects and, where molecular defects are present, identifying two principal molecular defects associated with the IGE;
4) comparing the identified molecular defects with pre-existing data correlating molecular defects with sub-syndromes of IGE; and
5) identifying the IGE.

19. An expression vector transformed with either a DNA molecule which encodes two mutant ion channel subunits, each containing a molecular defect or with two DNA molecules, each encoding an ion channel subunit with a molecular defect, in order that an animal in which the vector expresses protein has an IGE phenotype.

20. A host cell transformed by an expression vector as claimed in claim 19.

21. A genetically modified, non-human animal in which two ion channel subunits, each containing a molecular defect, are expressed in order that the animal has an IGE phenotype.

22. A genetically modified, non-human animal as claimed in claim 21 which has been transformed with a DNA molecule which encodes two ion channel subunits, each containing a molecular defect.

23. A genetically modified, non-human animal as claimed in claim 21 which has been transformed with two DNA molecules, each encoding an ion channel subunit with a molecular defect.

24. A genetically modified, non-human animal as claimed in any one of claims 21 to 23 which is selected from the group consisting of rats, mice, hamsters, guinea pigs, rabbits, dogs, cats, goats, sheep, pigs, and non-human primates such as monkeys and chimpanzees.

25. A method of producing a non-human transgenic animal containing a combination of two ion channel mutations comprising the steps of:
1) creating a non-human transgenic animal containing a mutation in a single ion channel subunit;
2) creating a second, non-human, transgenic animal containing a mutation in a different ion channel subunit; and
3) conducting mating combinations so as to produce progeny containing combinations of two ion channel mutations which effectively mimic combinations of ion channel mutations responsible for human IGE cases.

26. A non-human, transgenic animal produced by the process of claim 25.

27. The use of a genetically modified non-human animal as claimed in any one of claims 21 to 24, a non-human transgenic animal as claimed in claim 26 or a host cell as claimed in claim 20 for the screening of candidate pharmaceutical compounds.

28. A method for the treatment of an IGE, comprising administering to a patient with two principal molecular defects in their ion channel subunits which are causative of the IGE, one or more therapeutic agents which, singly or collectively, overcome or ameliorate the effect of the two principal molecular defects.

29. A method as claimed in claim 28 wherein the or each therapeutic agent is selected from the group consisting of a wild-type ion channel subunit polypeptide, a nucleic acid encoding a wild-type ion channel subunit, a nucleic acid encoding the complement of an ion channel subunit gene, and an agonist, modulator or antagonist of an ion channel subunit containing one of the two principal molecular defects.

30. Use of a wild-type ion channel subunit polypeptide, a nucleic acid encoding a wild-type ion channel subunit, a nucleic acid encoding the complement of an ion channel subunit gene, or an agonist, modulator or antagonist of an ion channel subunit containing one of two principal molecular defects causative of an IGE, in the preparation of a medicament for the treatment of the IGE.

31. A method of screening for therapeutic agents useful in the treatment of an IGE, wherein two principal molecular defects in the ion channel subunits of a subject are causative of the IGE, comprising introducing a potential therapeutic agent to a model system in which interaction with the two principal molecular defects is possible, and establishing which therapeutic agents overcome or ameliorate the effect of the two principal defects.
